(19) United States
(12) Patent Application Publication
(10) Pub. No.: US 2021/0214709 A1
(43) Pub. Date: Jul. 15, 2021

(54) COATINGS OF ENZYME PARTICLES COMPRISING ORGANIC WHITE PIGMENTS

(71) Applicant: BASF SE, Ludwigshafen (DE)

(72) Inventors: Ullrich Menge, Grenzach-Wyhlen (DE); Stefan Fischer, Ludwigshafen (DE)

(21) Appl. No.: 16/762,249

(22) PCT Filed: Oct. 30, 2018
(86) PCT No.: PCT/EP2018/079636

§ 371 Date: May 7, 2020

(30) Foreign Application Priority Data
Nov. 9, 2017 (EP) 17200746.0

(51) Int. Cl. C12N 9/98; C08L 71/02; C08L 25/06; C11D 3/386; C11D 3/40; A23L 29/00; A23L 5/47; A23K 20/189; A23K 20/179

(52) U.S. Cl. CPC C12N 9/98; C08L 71/02; C08L 25/06; C11D 3/38672; A23V 2002/00; A23L 29/06; A23L 5/47; A23K 20/189; A23K 20/179; C11D 3/40

(57) ABSTRACT

The present invention relates to novel enzyme particles comprising a core and a coating, wherein the core comprises at least one enzyme and the coating comprises at least one organic white pigment and uses thereof.

The present invention relates to novel enzyme particles wherein the coating comprises an organic white pigment. Moreover, the invention relates to processes for preparing the enzyme particles, and to their use in washing, cleaning, food, or feed compositions.

Inorganic whitening agents, such as titanium dioxide (TiO₂) are widely used as whitening agent. Especially titanium dioxide however is currently under suspicion of being a health risk. Therefore, an alternative whitening agent to inorganic whitening agents such as titanium dioxide for several whitening applications is needed.

Exemplarily in powder detergent enzyme technology, a whitening agent is needed to improve the whiteness of the final product. The search for efficient detergent compositions has been the object of research since a long time. The use of enzymes in detergent compositions is known and allows employing lower temperature and shorter periods of agitation. In general, enzyme detergents remove proteins from clothes soiled with e.g., blood, milk, sweat, or grass far more efficient than non-enzyme detergents. However, residual fermentation by-products lead to a dark color of the enzyme particle (i.e. of the final detergent powder). This is not acceptable from a consumer perspective. For this reason, white pigments are contained in the coating. A further issue of enzyme containing powder detergent compositions is the finding that human contact with airborne enzyme dust can cause severe allergic reactions. Therefore, producers and users may develop hypersensitivity. Thus, it is important to keep enzyme dust within acceptable levels and to provide abrasion resistance of the detergent enzyme particles. A further example of the application of a whitening agent for particles comprising enzymes is within the food and/or feed technology. The function of these enzymes is often to improve the feed conversion rate, e.g., by reducing the viscosity or by reducing the anti-nutritional effect of certain feed compounds. Feed enzymes can also be used to reduce the amount of compounds which are harmful to the environment in the manure. The food and/or feed enzyme compositions should be easily obtainable as well as easily compatible with usual food and/or feed ingredients. In addition, the compositions, especially the solid compositions should be easily processible, e.g. provide low dusting properties, be easy dispersible or mixable in the matrix desired.

Currently, inorganic solids, like zeolithe, kaolin (such as china clay), talkum, silica, and most preferably TiO₂ are used as white pigment in coatings of enzyme particles. Several attempts have been undertaken to substitute TiO₂ with other technologies. Exemplarily, WO 2015/028567 relates to enzyme containing granules comprising a fluorescent whitening agent for the use in detergents. It was found, that coatings for enzyme granules, comprising fluorescent whitening agents were efficient as substitutes of pigments, such as TiO₂. Depending on the applied fluorescent whitening agent the use may, however, result in health risks, as well. It is further not desirable to introduce fluorescent agents to the sewage in larger quantities.

WO 00/40689 relates to low-density compositions including enzyme particles, which are of relevance for especially liquid detergent compositions due to improved density properties. It is further described that these particles, having a layer structure with a non-pareil core and coatings, including borosilicate glass forming hollow spheres and TiO₂ as whitening agent, have low dusting levels.

In view of the above, it is an object of the present invention to provide an enzyme particle with improved whiteness.

In view of the above, it is an object of the present invention to provide an enzyme particle with alternative whitening agents.

It is a further object of the present invention to provide an enzyme particle with improved abrasion resistance.

It is a further object of the present invention to provide an enzyme particle including alternative agents to obtain abrasion resistance.

It is a further object of the present invention to provide a washing or cleaning composition, comprising enzyme particles, with improved whiteness and/or abrasion resistance.

It is a further object of the present invention to provide a food or feed compositing, comprising enzyme particles, with improved abrasion resistance.

At least one of the above addressed objects and others have been solved by coating an organic white pigment as defined herein onto an enzyme containing particle, where such particles are used for washing, cleaning, food, and feed compositions. In one embodiment, the enzyme is therefore a detergent enzyme. In another embodiment, the enzyme is therefore a food or feed enzyme.

In a first aspect, the present invention relates to an enzyme particle comprising a core and a coating, wherein the core comprises at least one enzyme and the coating comprises at least one organic white pigment.

In a second aspect, the present invention relates to a washing or cleaning composition, comprising enzyme particles according to the present invention.

In a third aspect, the present invention relates to a food or feed composition, comprising enzyme particles according to the present invention.

In a fourth aspect, the present invention relates to the use of at least one organic white pigment in the coating of enzyme particles.

In a further aspect of the invention, the organic white pigment is based on emulsion polymer particles, e.g., hollow emulsion polymer particles. In yet another aspect of the invention, the organic white pigment comprises polystyrene. In yet another aspect of the invention, the organic white pigment comprises polymethyl urea. Preferably, the organic white pigments comprise polystyrene or polymethyl urea.

It has surprisingly been found that the use of organic white pigments in the coating of enzyme containing particles improve the whiteness and/or the abrasion stability of said enzyme containing particles. It has further been found that the enzyme particles according to the present invention are suitable for the use in washing, cleaning, food, and/or feed compositions.

### Organic White Pigment

Within the meaning of this invention, the term organic white pigment is an organic pigment resulting in a white appearance. This white appearance can be measured by the following method.

A color paste is prepared by initially charging a vessel with 185 g of water and subsequently with the following ingredients, added in the stated order under a dissolver at about 1000 rpm and stirred homogeneous for altogether about 15 minutes: 2 g of 20 wt.-% aqueous sodium hydroxide solution, 12 g of Pigmentverteiler® MD 20 pigment disperser (copolymer of maleic acid and diisobutylene from BASF SE), 6 g of Agitan® E 255 (siloxane defoamer from Munzing Chemie GmbH), 725 g of Acronal® A 684 (binder, 50 wt.-% dispersion from BASF SE), 40 g of Texanol® (film-forming assistant from Eastman Chemical Company), 4 g of Agitan® E 255 (siloxane defoamer from Munzing Chemie GmbH), 25 g of DSX 3000 (30 wt.-%, associative thickener: hydrophobic modified polyether (HMPE)) and 2 g of DSX 3801 (45 wt.-%, associative thickener: hydrophobic modified ethoxylated urethane (HEUR)).

A 6 g quantity of the above described color paste and 0.312 g based on solids of the organic white pigment, which may be provided as dispersion, are weighed out into a vessel, the mixture is homogenized without stirring air thereinto. A 200 μm knife coater is used to draw down a film of this mixture on a black polymeric foil (matte option, article No. 13.41 EG 870934001, Bernd Schwegmann GmbH & Co. KG, D) at a speed of 0.9 cm/sec. The samples are dried at 23° C. and a relative humidity of 40-50% for 24 h. Subsequently, a Minolta CM-508i spectrophotometer is used to measure the whiteness (L-value from L*a*b color space to EN ISO 11664-4:2012-06) at three different places. The places where the measurements are carried out are marked in order that a micrometer screw may subsequently be used to determine the corresponding thicknesses of the colored-film layer by differential measurement relative to the uncoated polymeric foil. After computing an average film thickness and also an average whiteness from the three individual measurements, the whiteness level obtained is finally standardized to a dry film thickness of 50 μm by linear extrapolation. The calibration needed for this is done by measuring the whiteness of a standard organic white pigment in a dry film thickness range of about 30-60 μm. In one embodiment, the organic white pigment according to the present invention exhibits an L-value of at least 70, or at least 78, or at least 85 or at least 90, or at least 95. In one embodiment, the organic white pigment according to the present invention exhibits an L-value of from 70 to 95, or of from 75 to 85.

If not otherwise indicated, the particle sizes of the organic white pigment are determined by hydrodynamic fractionation using a Polymer Labs particle size distribution analyzer (PSDA). The Cartridge PL0850-1020 column used is operated with a flow rate of 2 ml·min⁻¹. The samples are diluted with eluent solution down to an absorption of 0.03 AU·μl⁻¹.

The sample is eluted by the size exclusion principle according to the hydrodynamic diameter. The eluent comprises 0.2 wt.-% of dodecylpoly(ethylene glycol ether)₂₃, 0.05 wt.-% of sodium dodecylsulfonate, 0.02 wt.-% of sodium dihydrogen phosphate and 0.02 wt.-% of sodium azide in deionized water. The pH is 5.8. The elution time is calibrated with polystyrene calibration lattices. The measurement range extends from 20 nm to 1200 nm. Detection is by UV detector at wavelength 254 nm.

Particle size can further be determined using a Coulter M4+ Particle Analyzer or by photon correlation spectroscopy also known as quasi elastic light scattering or dynamic light scattering (DIN ISO 13321:2004-10) using a Malvern high performance particle sizer (HPPS).

It is to be understood that pigments change the color of reflected or transmitted light as the result of selective absorption of certain wavelengths. This physical process thus differs fundamentally from e.g. fluorescence, in which a material emits light. Thus, pigments and fluorescent agents are dissimilar materials, exhibiting different properties, and are underlying different physical principles. Within the meaning of the present invention, the organic white pigment is not a fluorescent whitening agent.

In one embodiment, the at least one organic white pigment is in the form of organic particles, in particular hollow organic particles.

In one embodiment, the at least one organic white pigment is based on polymers, comprising nonionic ethylenically unsaturated monomers. Preferably, the nonionic ethylenically unsaturated monomer is selected from the group consisting of styrene, acrylonitrile, methacrylamide, methyl methacrylate, ethyl acrylate, ethyl methacrylate, butyl acrylate, butyl methacrylate, 2-ethylhexyl acrylate, 2-ethylhexyl methacrylate, or mixtures thereof. In one embodiment, the at least one organic white pigment is based on a polymer comprising styrene. Preferably, the at least one organic white pigment is a polystyrene particle. More preferably, the at least one organic white pigment is a hollow sphere based on a polymer comprising styrene.

The at least one organic white pigment may be applied to the enzyme containing core as dispersion having a solid content of at least 10 wt.-%. In a certain embodiment, the solid content of the dispersion is less than 50 wt.-%. In another certain embodiment, the solid content of the dispersion between 10 and 50 wt.-%.

In one embodiment, the organic white pigment is based on emulsion polymer particles obtained by a process as described in US 2001009929. In accordance with the disclosure of US 2001009929 in a certain embodiment, the organic white pigments are based on emulsion polymer particles obtainable by a process for preparing emulsion polymer particles comprising:
a) providing an aqueous emulsion of
   i) multi-stage emulsion polymer, comprising a core stage polymer and a shell stage polymer (herein defined as sheath stage polymer),
      wherein the core stage polymer comprises, as polymerized units, from 5 to 100 percent by weight, based on the weight of the core stage polymer, of hydrophilic monoethylenically unsaturated monomer (herein defined as hydrophilic ethylenically unsaturated monomer), and from 0 to 95 percent by weight, based on the weight of the core stage polymer, of at least one nonionic monoethylenically unsaturated monomer (herein defined as nonionic ethylenically unsaturated monomer); and wherein the shell stage polymer comprises, as polymerized units, at least 50 percent by weight of nonionic monoethylenically unsaturated monomer;
   ii) monomer at a level of at least 0.5 percent by weight based on the weight of the multi-stage emulsion polymer; and
   iii) swelling agent
   and
b) reducing the level of monomer by at least fifty percent.

In another embodiment, the organic white pigments are based on emulsion polymer particles obtainable by a process for preparing emulsion polymer particles comprising:
a) providing an aqueous emulsion of
   i) multi-stage emulsion polymer, comprising a core stage polymer and a shell stage polymer (herein defined as sheath stage polymer),
      wherein the core stage polymer comprises, as polymerized units, from 5 to 100 percent by weight, based on the weight of the core stage polymer, of hydrophilic monoethylenically unsaturated monomer (herein defined as hydrophilic ethylenically unsaturated monomer), and from 0 to 95 percent by weight, based on the weight of the core stage polymer, of at least one nonionic monoethylenically unsaturated monomer (herein defined as nonionic ethylenically unsaturated monomer); and wherein the shell stage polymer comprises, as polymerized units, at least 50 percent by weight of nonionic monoethylenically unsaturated monomer;
   ii) monomer at a level of at least 0.5 percent by weight based on the weight of the multi-stage emulsion polymer; and
   iii) swelling agent under conditions wherein there is no substantial polymerization of the monomer; and
b) reducing the level of monomer by at least fifty percent.

In another embodiment, the organic white pigment is based on emulsion polymer particles obtained by a process as described in US2012245240A. In accordance with the disclosure of US2012245240A in a certain embodiment, the organic white pigments are based on emulsion polymer particles obtainable by a process for preparing an emulsion containing core-sheath-shell polymer particles, without any polymerization inhibitors or scavengers, said process comprising the steps of:
(i) emulsion polymerizing a core (A) from a core monomer system comprising, as polymerized units, from about 5% to about 100% by weight, based on the weight of the core, of hydrophilic monoethylenically unsaturated monomer containing acid functionality (herein defined as hydrophilic ethylenically unsaturated monomer), and from about 0 to about 95% by weight, based on the weight of the core, of at least one nonionic monoethylenically unsaturated monomer (herein defined as nonionic ethylenically unsaturated monomer);
(ii) encapsulating said core (A) with a sheath polymeric layer (B) by emulsion polymerizing a sheath monomer system (E1) comprising, as polymerized units, at least about 20% by weight of a hydrophilic monoethylenically unsaturated monomer, at least about 20% by weight of a hydrophobic monoethylenically unsaturated monomer, and about 1% to about 20% by weight of a hydrophilic monoethylenically unsaturated monomer containing acid functionality, each based on the total weight of the sheath polymeric layer, in the presence of said core, said sheath permitting penetration of volatile, fixed or permanent bases;
(iii) encapsulating said core-sheath particles with a polymeric shell (C) by emulsion polymerizing a shell monomer system (E2) comprising, as polymerized units, from about 1% to about 10% by weight, of hydrophilic monoethylenically unsaturated monomer containing acid functionality, and from about 90% to about 99% by weight, of at least one nonionic monoethylenically unsaturated monomer, each based on the total weight of the polymeric shell;
(iv) neutralizing and swelling, at elevated temperature, the resultant core-sheath-shell polymer particles with a volatile, fixed or permanent base, said swelling taking place in the presence of a monomer-solvent-system comprising from about 5% to about 50% by weight of the at least one nonionic monoethylenically unsaturated monomer of said shell monomer system (E2), wherein said monomer-solvent-system is added before, after, or during the addition of the base, and
(v) after the swelling step, reducing the level of said at least one nonionic monoethylenically unsaturated monomer of said monomer-solvent-system in step (iv) by polymerizing the monomer to less than about 10,000 ppm, based on polymer solids, so as to produce an emulsion of particles which, when dried, contain a microvoid which causes opacity in compositions in which they are contained, wherein a water soluble polymerization catalyst in a total amount of about 0.05% to about 0.45% by weight, based on the total amount of monomers in E1 and E2, is either fed in parallel with the sheath monomer system E1 into the polymerization reactor or is fed into the polymerization reactor before emulsion polymerization of E1 in step (ii) starts.

Suitable swelling agents include those which, in the presence of the multi-stage emulsion polymer and monomer, are capable of permeating the shell and swelling the core. Swelling agents may be aqueous or gaseous, volatile or fixed bases or combinations thereof.

Suitable swelling agents include volatile bases such as ammonia, ammonium hydroxide, preferably aqueous ammonium hydroxide, and volatile lower aliphatic amines, such as morpholine, trimethylamine, and triethylamine, and the like; fixed or permanent bases such as potassium hydroxide, lithium hydroxide, zinc ammonium complex, copper ammonium complex, silver ammonium complex, strontium hydroxide, barium hydroxide and the like. Sodium hydroxide and potassium hydroxide are preferred.

In another embodiment, the organic white pigment is based on emulsion polymer particles obtained by a process as described in WO 2015024882. In accordance with the disclosure of WO 2015024882 in a certain embodiment, the organic white pigments are based on emulsion polymer particles obtainable by producing a multistaged emulsion polymer by
i) polymerizing in a sequential polymerization a seed,
ii) then reacting with a swell-seed comprising 55 to 99.9 wt.-% of one or more than one nonionic ethylenically unsaturated monomer and 0.1 to 45 wt.-% of one or more than one ethylenically unsaturated hydrophilic monomer, all based on the overall weight of the core stage polymer comprising both seed and swell-seed,
iii) then polymerizing a first shell comprising 85 to 99.9 wt.-% of one or more than one nonionic ethylenically unsaturated monomer and 0.1 to 15 wt.-% of one or more than one hydrophilic ethylenically unsaturated monomer,
iv) then polymerizing a second shell comprising 85 to 99.9 wt.-% of one or more than one nonionic ethylenically unsaturated monomer and 0.1 to 15 wt.-% of one or more than one hydrophilic ethylenically unsaturated monomer,
v) then adding at least one plasticizer monomer having a ceiling temperature below 181° C., preferably below 95° C.,
vi) neutralizing, to a pH of not less than 7.5, the resultant particles with a base,
vii) then polymerizing a third shell comprising 90 to 99.9 wt.-% of one or more than one nonionic ethylenically unsaturated monomer and 0.1 to 10 wt.-% of one or more than one hydrophilic ethylenically unsaturated monomer,
viii) and also optionally polymerizing one or more further shells comprising one or more than one nonionic ethylenically unsaturated monomer and one or more than one hydrophilic ethylenically unsaturated monomer, wherein
   the weight ratio of said swell-seed (ii) to said seed polymer (i) is in the range from 10:1 to 150:1, the weight ratio of the core stage polymer to said first shell (iii) is in the range from 2:1 to 1:5, and the weight ratio of said third shell (vii) to said second shell (iv) is in the range from 1:2 to 1:10.

In another embodiment, the organic white pigment is based on emulsion polymer particles obtained by a process as described in WO 2015024835. In accordance with the disclosure of WO 2015024835 in a certain embodiment, the at least one organic white pigment consists of at least one hollow organic particle, based on emulsion polymer particles obtainable by the method comprising
(i) performing a sequential polymerization to obtain a multistaged emulsion polymer in the form of particles;
ii) neutralizing the particles with at least one base up to a pH of not less than 7.5; and
iii) optionally polymerizing further shells comprising one or more nonionic ethylenically unsaturated monomer, wherein
   the multistaged emulsion polymer comprises at least a core stage polymer and a sheath stage polymer; the core stage polymer comprises by way of polymerized units from 5 to 99.5 wt.-%, based on the weight of the core stage polymer, of at least one hydrophilic ethylenically unsaturated monomer, from 0 to 95 wt.-%, based on the weight of the core stage polymer, of at least one nonionic ethylenically unsaturated monomer, and 0.5 to 20 wt.-% of at least one nonionic polyalkylene oxide containing additive, based on the weight of the core stage polymer; and the sheath stage polymer comprises by way of polymerized units not less than 50 wt.-% of a nonionic ethylenically unsaturated monomer.

In another certain embodiment, the at least one organic white pigment (comprised in the coating of the enzyme particle) consists of at least one hollow organic particle, based on emulsion polymer particles, obtainable by sequential polymerization, comprising polymerizing in a sequential polymerization
i) a seed, and
ii) then reacting with a swell-seed comprising 55 to 99.9 wt.-% of one or more nonionic ethylenically unsaturated monomer and 0.1 to 45 wt.-% of one or more ethylenically unsaturated hydrophilic monomer, all based on an overall weight of a core stage polymer comprising both seed and swell-seed,
iii) then polymerizing a first shell comprising 85 to 99.9 wt.-% of one or more than nonionic ethylenically unsaturated monomer and 0.1 to 15 wt.-% of one or more hydrophilic ethylenically unsaturated monomer,
iv) then polymerizing a second shell comprising 85 to 99.9 wt.-% of one or more nonionic ethylenically unsaturated monomer and 0.1 to 15 wt.-% of one or more hydrophilic ethylenically un-saturated monomer,
v) then adding at least one plasticizer monomer having a ceiling temperature below 181° C.,
vi) neutralizing, to a pH of not less than 7.5 or greater, the resultant particles with one or more bases,
vii) then polymerizing a third shell comprising 90 to 99.9 wt.-% of one or more nonionic ethylenically unsaturated monomer and 0.1 to 10 wt.-% of one or more hydrophilic ethylenically unsaturated monomer,
viii) also optionally polymerizing one or more further shells comprising one or more nonionic ethylenically unsaturated monomer and one or more hydrophilic ethylenically unsaturated monomer, wherein
   a weight ratio of the swell-seed (ii) to the seed polymer (i) is in a range from 10:1 to 150:1, a weight ratio of the core stage polymer to the first shell (iii) is in a range from 2:1 to 1:5, and a weight ratio of the third shell (vii) to the second shell (iv) is in a range from 1:2 to 1:10.

The process to obtain emulsion polymer particles (which are the basis to certain organic white pigments) is a multistaged sequential emulsion polymerization. Sequential relates to the implementation of the individual stages in that each individual stage may also be constructed of two or more sequential steps.

An organic white pigment according to the present invention may be obtained by drying (e.g. during spray coating) an emulsion polymer particle obtained as described above and discussed in more detail below. The typical residual moisture of the final coated enzyme particle is thus less than 15 wt.-%, preferably less than 5 wt.-%, most preferable less than 2 wt.-%.

The term “seed” refers to an aqueous polymeric dispersion which is used at the start of the multistaged polymerization and is the product of an emulsion polymerization, or to an aqueous polymeric dispersion present at the end of one of the polymerization stages for producing the hollow particle dispersion, except the last stage.

The seed used at the start of polymerizing the first stage may also be formed in situ and preferably comprises as monomer constituents styrene, acrylic acid, methacrylic acid, esters of acrylic acid and methacrylic acid or mixtures thereof. Preferably, the seed used at the start of polymerizing the first stage is formed in situ and comprises as monomer constituents styrene.

The average particle size of the seed polymer in the unswollen state is in the range from 20 to 100 nm.

The swell-seed comprises 55 to 99.9 wt.-%, preferably 60 to 80 wt.-%, of a nonionic ethylenically unsaturated monomer and 0.1 to 45 wt.-%, preferably 20 to 40 wt.-%, of an ethylenically unsaturated hydrophilic monomer.

The weight ratio of swell-seed (ii) to seed polymer (i) is in the range from 10:1 to 150:1. The average particle size in the unswollen state of the core stage polymer consisting of seed (i) and swell-seed (ii) is in the range from 50 to 300 nm, preferably in the range from 50 to 200 nm. The glass transition temperature determined by the Fox equation (John Wiley & Sons Ltd., Baffins Lane, Chichester, England, 1997) of the core stage polymer in the protonated state is between −20° C. and 150° C.

The polyalkylene oxide nonionic additives may be selected from the group consisting of polysiloxane-polyalkylene oxide copolymers, such as polysiloxane-polyalkylene oxide graft copolymers of comb structure, polysiloxane-polyalkylene oxide graft copolymers of α,ω structure, polysiloxane-polyalkylene oxide graft copolymers having ABA or BAB block structures or further sequences of polyalkylene oxide polysiloxane blocks, branched polysiloxane-polyalkylene oxide copolymers, polysiloxane-polyalkylene oxide graft copolymers having polyester, (fluorinated) (poly)alkyl, polyacrylate side chains; copolymers of propylene oxide, butylene oxide or styrene oxide and ethylene oxide, block copolymers of propylene oxide and ethylene oxide, polyalkylene oxide-poly(meth)acrylate copolymers, polyalkylene oxide-(poly)alkyl copolymers, poly(alkylene oxide)-poly((meth)acrylate) block copolymer, fluorinated alkyl ester polyalkylene oxides and polyalkoxylates and highly branched polyalkylene oxides, preferably polysiloxanepolyalkylene oxide graft copolymers of comb structure, or mixtures thereof.

Nonionic ethylenically unsaturated monomers are for example styrene, vinyltoluene, ethylene, butadiene, vinyl acetate, vinyl chloride, vinylidene chloride, acrylonitrile, acrylamide, methacrylamide, (C₁-C₂₀)alkyl or (C₃-C₂₀)alkenyl esters of acrylic or methacrylic acid, methyl methacrylate, ethyl acrylate, ethyl methacrylate, butyl acrylate, butyl methacrylate, 2-ethylhexyl acrylate, 2-ethylhexyl methacrylate, benzyl acrylate, benzyl methacrylate, lauryl acrylate, lauryl methacrylate, oleyl acrylate, oleyl methacrylate, palmityl acrylate, palmityl methacrylate, stearyl acrylate, stearyl methacrylate, hydroxyl-containing monomers, in particular C₁-C₁₀ hydroxyalkyl (meth)acrylates, such as hydroxyethyl (meth)acrylate, hydroxypropyl (meth)acrylate, glycidyl (meth)acrylate, preferably methyl methacrylate. Most preferably, the nonionic ethylenically unsaturated monomers is styrene.

Ethylenically unsaturated hydrophilic monomers are for example acrylic acid, methacrylic acid, acryloyloxypropionic acid, methacryloyloxypropionic acid, acryloyloxyacetic acid, methacryloyloxyacetic acid, crotonic acid, aconitic acid, itaconic acid, monomethyl maleate, maleic acid, monomethyl itaconate, maleic anhydride, fumaric acid, monomethyl fumarate, itaconic anhydride, and also linseed oil fatty acids, such as oleic acid, linoleic acid and linolenic acid and also further fatty acids, such as ricinoleic acid, palmitoleic acid, elaidic acid, vaccenic acid, icosenic acid, cetoleic acid, erucic acid, nervonic acid, arachidonic acid, timnodonic acid, clupanodonic acid, preferably acrylic acid and methacrylic acid.

In one embodiment, the sheath stage polymer comprises not less than 50 wt.-% of a nonionic ethylenically unsaturated monomer.

The nonionic ethylenically unsaturated monomers of the sheath stage polymer are for example styrene, ethylvinylbenzene, vinyltoluene, ethylene, butadiene, vinyl acetate, vinyl chloride, vinylidene chloride, acrylonitrile, acrylamide, methacrylamide, (C1-C20)alkyl or (C3-C20)alkenyl esters of acrylic or methacrylic acid, methyl methacrylate, ethyl acrylate, ethyl methacrylate, butyl acrylate, butyl methacrylate, 2-ethylhexyl acrylate, 2-ethylhexyl methacrylate, benzyl acrylate, benzyl methacrylate, lauryl acrylate, lauryl methacrylate, oleyl acrylate, oleyl methacrylate, palmityl acrylate, palmityl methacrylate, stearyl acrylate, stearyl methacrylate, hydroxyl-containing monomers, in particular C1-C10 hydroxyalkyl (meth)acrylates, such as hydroxyethyl (meth)acrylate, hydroxypropyl (meth)acrylate, glycidyl (meth)acrylate, preferably styrene, acrylonitrile, methacrylamide, methacrylate, methyl methacrylate, ethyl acrylate, ethyl methacrylate, butyl acrylate, butyl methacrylate, 2-ethylhexyl acrylate, 2-ethylhexyl methacrylate. Most preferably, the nonionic ethylenically unsaturated monomer of the sheath stage polymer is styrene.

The sheath stage polymer encloses the core stage polymer and in the protonated state has a glass transition temperature, determined by the Fox equation, of between −60 and 120° C. The particle size of core-shell polymer, consisting of core stage and sheath stage polymer in the unswollen state, is in the range from 60 nm to 1000 nm and preferably in the range from 60 to 500 nm.

The first shell (iii) comprises 85 to 99.9 wt.-% of one or more than one nonionic ethylenically unsaturated monomer, preferably 90 to 99.9 wt.-%, and also 0.1 to 15 wt.-%, preferably 0.1 to 10 wt.-% of one or more than one hydrophilic ethylenically unsaturated monomer.

Nonionic ethylenically unsaturated monomers are for example styrene, vinyltoluene, ethylene, butadiene, vinyl acetate, vinyl chloride, vinylidene chloride, acrylonitrile, acrylamide, methacrylamide, (C₁-C₂₀)alkyl or (C₃-C₂₀)alkenyl esters of acrylic or methacrylic acid, methyl methacrylate, ethyl acrylate, ethyl methacrylate, butyl acrylate, butyl methacrylate, 2-ethylhexyl acrylate, 2-ethylhexyl methacrylate, benzyl acrylate, benzyl methacrylate, lauryl acrylate, lauryl methacrylate, oleyl acrylate, oleyl methacrylate, palmityl acrylate, palmityl methacrylate, stearyl acrylate, stearyl methacrylate, hydroxyl-containing monomers, in particular C₁-C₁₀ hydroxyalkyl (meth)acrylates, such as hydroxyethyl (meth)acrylate, hydroxypropyl (meth)acrylate, glycidyl (meth)acrylate, preferably styrene, acrylonitrile, methacrylamide, methyl methacrylate, ethyl acrylate, ethyl methacrylate, butyl acrylate, butyl methacrylate, 2-ethylhexyl acrylate, 2-ethylhexyl methacrylate. Most preferably, the nonionic ethylenically unsaturated monomer is styrene.

Ethylenically unsaturated hydrophilic monomers are for example acrylic acid, methacrylic acid, acryloyloxypropionic acid, methacryloyloxypropionic acid, acryloyloxyacetic acid, methacryloyloxyacetic acid, crotonic acid, aconitic acid, itaconic acid, monomethyl maleate, maleic acid, monomethyl itaconate, maleic anhydride, fumaric acid, monomethyl fumarate, and also linseed oil fatty acids, such as oleic acid, linoleic acid and linolenic acid and also further fatty acids, such as ricinoleic acid, palmitoleic acid, elaidic acid, vaccenic acid, icosenic acid, cetoleic acid, erucic acid, nervonic acid, arachidonic acid, timnodonic acid, clupanodonic acid, preferably acrylic acid, methacrylic acid, itaconic acid, itaconic anhydride, monomethyl itaconate.

The first shell (iii) encloses the core stage polymer. The weight ratio of the core stage polymer to the first shell (iii) is in the range from 2:1 to 1:5 preferably 2:1 to 1:3, and the shell polymer in the protonated state has a glass transition temperature determined by the Fox equation between −60° C. to 120° C.

The particle size of this stage consisting of core stage polymer and first shell (iii) in the unswollen state is from 60 nm to 500 nm, preferably from 60 to 300 nm.

The second shell (iv) comprises 85 to 99.9, preferably 90 to 99.9 wt.-% of one or more than one nonionic ethylenically unsaturated monomer and 0.1 to 15 wt.-%, preferably 0.1 to 10 wt.-% of one or more than one hydrophilic ethylenically unsaturated monomer.

Nonionic ethylenically unsaturated monomers are for example styrene, p-methylstyrene, t-butylstyrene, vinyltoluene, ethylene, butadiene, vinyl acetate, vinyl chloride, vinylidene chloride, acrylonitrile, acrylamide, methacrylamide, (C₁-C₂₀)alkyl or (C₃-C₂₀)alkenyl esters of acrylic or methacrylic acid, methyl methacrylate, ethyl acrylate, ethyl methacrylate, butyl acrylate, butyl methacrylate, 2-ethylhexyl acrylate, 2-ethylhexyl methacrylate, benzyl acrylate, benzyl methacrylate, lauryl acrylate, lauryl methacrylate, oleyl acrylate, oleyl methacrylate, palmityl acrylate, palmityl methacrylate, stearyl acrylate, stearyl methacrylate, hydroxyl-containing monomers, in particular C₁-C₁₀ hydroxyalkyl (meth)acrylates, such as hydroxyethyl (meth)acrylate, hydroxypropyl (meth)acrylate, glycidyl (meth)acrylate, preferably styrene, acrylonitrile, methacrylamide, methyl methacrylate, ethyl acrylate, ethyl methacrylate, butyl acrylate, butyl methacrylate, 2-ethylhexyl acrylate, 2-ethylhexyl methacrylate. Most preferably, the nonionic ethylenically unsaturated monomer is styrene.

Ethylenically unsaturated hydrophilic monomers are for example acrylic acid, methacrylic acid, acryloyloxypropionic acid, methacryloyloxypropionic acid, acryloyloxyacetic acid, methacryloyloxyacetic acid, crotonic acid, aconitic acid, itaconic acid, monomethyl maleate, maleic acid, monomethyl itaconate, maleic anhydride, fumaric acid, monomethyl fumarate, and also linseed oil fatty acids, such as oleic acid, linoleic acid and linolenic acid and also further fatty acids, such as ricinoleic acid, palmitoleic acid, elaidic acid, vaccenic acid, icosenic acid, cetoleic acid, erucic acid, nervonic acid, arachidonic acid, timnodonic acid, clupanodonic acid, preferably acrylic acid, methacrylic acid, itaconic acid, itaconic anhydride, monomethyl itaconate and linseed oil fatty acids.

The first shell is enveloped by the second shell and the weight ratio of the first shell (iii) to the second shell (iv) is in the range from 1:1 to 1:30, and the shell polymer in the protonated state has a Fox glass transition temperature of 50 to 120° C.

The average particle size of this stage, consisting of core stage polymer, first shell (iii) and second shell (iv), in the unswollen state is in the range from 70 to 1000 nm.

The plasticizer monomer recited under (v) is for example α-methylstyrene, esters of 2-phenylacrylic acid/atropic acid (e.g., methyl, ethyl, n-propyl, n-butyl), 2-methyl-2-butene, 2,3-dimethyl-2-butene, 1,1-diphenylethene or methyl 2-tert-butylacrylate, and also further monomers recited in J. Brandrup, E. H. Immergut, Polymer Handbook 3rd Edition, II/316ff. α-Methylstyrene is preferably used as plasticizer monomer.

When the polymerization is carried out in aqueous solution or dilution, the monomers may be wholly or partly neutralized with bases before or during the polymerization. Useful bases include for example alkali metal or alkaline earth metal compounds such as sodium hydroxide, potassium hydroxide, calcium hydroxide, magnesium oxide, sodium carbonate; ammonia; primary, secondary and tertiary amines, such as ethylamine, propylamine, monoisopropylamine, monobutylamine, hexylamine, ethanolamine, dimethylamine, diethylamine, di-n-propylamine, tributylamine, triethanolamine, dimethoxyethylamine, 2-ethoxyethylamine, 3-ethoxypropylamine, dimethylethanolamine, diisopropanolamine, morpholine, ethylenediamine, 2-diethylamino-ethylamine, 2,3-diaminopropane, 1,2-propylenediamine, dimethylaminopropylamine, neopentanediamine, hexamethylenediamine, 4,9-dioxadodecane-1,12-diamine, polyethylene-imine, polyvinylamine or mixtures thereof.

The ethylenically unsaturated hydrophilic monomers used in (i)-(v) are preferably not neutralized before or during the polymerization.

The neutralization recited under (vi) is effected with one or more of the illustratively recited bases for swelling the core and hence leads to the formation of the hollow organic particle after drying.

It is preferable to use sodium hydroxide, ammonia, triethanolamine and diethanolamine for the neutralization recited under (vi). The ethylenically unsaturated hydrophilic monomers used after (vi) are preferably neutralized during the polymerization.

The third shell (vii) comprises 90 to 99.9, preferably 95 to 99.9 wt.-% of one or more than one nonionic ethylenically unsaturated monomer and 0.1 to 10, preferably 0.1 to 5 wt.-% of one or more than one hydrophilic ethylenically unsaturated monomer.

The nonionic ethylenically unsaturated monomers are for example styrene, ethylvinylbenzene, vinyltoluene, ethylene, butadiene, vinyl acetate, vinyl chloride, vinylidene chloride, acrylonitrile, acrylamide, methacrylamide, (C₁-C₂₀)alkyl or (C₃-C₂₀)alkenyl esters of acrylic or methacrylic acid, methyl methacrylate, ethyl acrylate, ethyl methacrylate, butyl acrylate, butyl methacrylate, 2-ethylhexyl acrylate, 2-ethylhexyl methacrylate, benzyl acrylate, benzyl methacrylate, lauryl acrylate, lauryl methacrylate, oleyl acrylate, oleyl methacrylate, palmityl acrylate, palmityl methacrylate, stearyl acrylate, stearyl methacrylate, hydroxyl-containing monomers, in particular C₁-C₁₀ hydroxyalkyl (meth)acrylates, such as hydroxyethyl (meth)acrylate, hydroxypropyl (meth)acrylate, glycidyl (meth)acrylate, preferably styrene, acrylonitrile, methacrylamide, methyl methacrylate, ethyl acrylate, ethyl methacrylate, butyl acrylate, butyl methacrylate, 2-ethylhexyl acrylate, 2-ethylhexyl methacrylate. Most preferably, the nonionic ethylenically unsaturated monomer is styrene.

The ethylenically unsaturated hydrophilic monomers are for example acrylic acid, methacrylic acid, acryloyloxypropionic acid, methacryloyloxypropionic acid, acryloyloxyacetic acid, methacryloyloxyacetic acid, crotonic acid, aconitic acid, itaconic acid, monomethyl maleate, maleic acid, monomethyl itaconate, maleic anhydride, fumaric acid, monomethyl fumarate, and also linseed oil fatty acids, such as oleic acid, linoleic acid and linolenic acid and also further fatty acids, such as ricinoleic acid, palmitoleic acid, elaidic acid, vaccenic acid, icosenic acid, cetoleic acid, erucic acid, nervonic acid, arachidonic acid, timnodonic acid, clupanodonic acid, preferably acrylic acid, methacrylic acid, itaconic acid, itaconic anhydride, monomethyl itaconate and linseed oil fatty acids.

The weight ratio of third to second shell is in the range from 1:2 to 1:10, and the shell polymer has a Fox glass transition temperature of 50 to 120° C.

The average final particle size of the polymers ranges from 100 to 10000 nm, preferably from 100 to 2500 nm.

The polymers are obtainable via customary methods of emulsion polymerization. It is preferable to operate in the absence of oxygen, e.g., in a stream of nitrogen. Customary apparatus is employed for the polymerization procedure, examples being stirred tanks, stirred-tank cascades, autoclaves, tubular reactors and kneaders. The polymerization can be carried out in solvent or diluent media, e.g., toluene, o-xylene, p-xylene, cumene, chlorobenzene, ethylbenzene, technical-grade mixtures of alkylaromatics, cyclohexane, technical-grade aliphatics mixtures, acetone, cyclohexanone, tetrahydrofuran, dioxane, glycols and glycol derivatives, polyalkylene glycols and derivatives thereof, diethyl ether, tert-butyl methyl ether, methyl acetate, isopropanol, ethanol, water or mixtures such as, for example, isopropanol-water mixtures.

The polymerization can be carried out at temperatures of 20 to 300° C., preferably of 50 to 200° C.

The polymerization is in one embodiment carried out in the presence of compounds that form free radicals. These compounds are needed in a proportion of up to 30 wt.-%, preferably 0.05 to 15 wt.-%, more preferably 0.2 to 8 wt.-%, based on the monomers used in the polymerization. In the case of multicomponent initiator systems (e.g., redox initiator systems), the foregoing weight particulars are based on total components. Useful polymerization initiators include, for example, peroxides, hydroperoxides, peroxodisulfates, percarbonates, peroxyesters, hydrogen peroxide and azo compounds. Examples of initiators, which can be water soluble or else water insoluble, are hydrogen peroxide, dibenzoyl peroxide, dicyclohexyl peroxydicarbonate, dilauroyl peroxide, methyl ethyl ketone peroxide, di-tert-butyl peroxide, acetylacetone peroxide, tert-butyl hydroperoxide, cumene hydroperoxide, tert-butyl perneodecanoate, tert-amyl perpivalate, tert-butyl perpivalate, tert-butyl perneohexanoate, tert-butyl per-2-ethyl hexanoate, tert-butyl perbenzoate, lithium peroxodisulfate, sodium peroxodisulfate, potassium peroxodisulfate, ammonium peroxodisulfate, azobisisobutyronitrile, 2,2′-azobis(2-amidinopropane) dihydrochloride, 2-(carbamoylazo)isobutyronitrile and 4,4-azobis(4-cyanovaleric acid).

The initiators may be used alone or mixed with each or one another, for example mixtures of hydrogen peroxide and sodium peroxodisulfate. Polymerization in an aqueous medium preferably utilizes water-soluble initiators.

The familiar redox initiator systems can also be used as polymerization initiators. Redox initiator systems of this type comprise one or more than one peroxide-containing compound combined with a redox co-initiator, e.g., sulfur compounds having a reducing effect, examples being bisulfites, sulfites, sulfinates, thiosulfates, dithionites and tetrathionates of alkali metals and ammonium compounds and their adducts such as sodium hydroxymethylsulfinates and acetone bisulfites and also ascorbic acid, isoascorbic acid and sodium erythrobate. Combinations of peroxodisulfates with alkali metal or ammonium hydrogensulfites can accordingly be used, an example being ammonium peroxodisulfate combined with ammonium disulfite. The ratio of peroxide-containing compound to redox co-initiator is in the range from 30:1 to 0.05:1.

Transition metal catalysts may additionally be used in combination with the initiators and/or the redox initiator systems, examples being salts of iron, cobalt, nickel, copper, vanadium and manganese. Useful salts include, for example, iron(II) sulfate, cobalt(II) chloride, nickel(II) sulfate, copper(I) chloride or else water-soluble iron-chelate complexes such as K[Fe-III-EDTA] or Na[Fe-Ill-EDTA]. Based on monomers, the reducing transition metal salt is used in a concentration of 0.1 to 1000 ppm. Combinations of hydrogen peroxide with iron(II) salts can accordingly be used, an example being 0.5 to 30% of hydrogen peroxide being combined with 0.1 to 500 ppm of Mohr's salt.

Similarly, polymerization in organic solvents may combine the abovementioned initiators with redox co-initiators and/or transition metal catalysts, examples being benzoin, dimethylaniline, ascorbic acid and also organosoluble complexes of heavy metals, such as copper, cobalt, iron, manganese, nickel and chromium. The customarily used amounts of redox co-initiators and/or transition metal catalysts are here customarily about 0.1 to 1000 ppm, based on the amounts of monomers used. When the reaction mixture is incipiently polymerized at the lower limit of the temperature range for the polymerization and then fully polymerized at a higher temperature, it is advantageous to use two or more different initiators that decompose at different temperatures, so an adequate concentration of free radicals is available within every temperature interval, or to use a redox initiator system wherein the peroxide-containing component is initially activated by a co-initiator at a low temperature and thermally decomposes at a higher temperature without a continued need for co-initiator.

The initiator can also be added in stages, and/or the rate of initiator addition varied over time. To obtain polymers of low average molecular weight, it is often advantageous to conduct the copolymerization in the presence of chain transfer agents. The chain transfer agents used for this may be customary chain transfer agents, for example organic SH-containing compounds, such as 2-mercaptoethanol, 2-mercaptopropanol, mercaptoacetic acid, tert-butyl mercaptan, n-octyl mercaptan, n-dodecyl mercaptan and tert-dodecyl mercaptan, C₁-C₄ aldehydes, such as formaldehyde, acetaldehyde, propionaldehyde, hydroxylammonium salts such as hydroxyl-ammonium sulfate, formic acid, sodium bisulfite, hypophosphorous acid and/or salts thereof, or isopropanol.

Chain transfer agents are generally used in amounts of 0.1 to 20 wt.-%, based on the monomers. The choice of a suitable solvent is another way to control the average molecular weight. Thus, polymerization in the presence of diluents having benzylic hydrogen atoms, or in the presence of secondary alcohols such as, for example, isopropanol, leads to a reduction in the average molecular weight through chain transfer.

Polymers of low or comparatively low molecular weight are also obtained through: varying the temperature and/or the initiator concentration and/or the monomer feed rate.

To obtain comparatively high molecular weight copolymers, it is often advantageous to perform the polymerization in the presence of crosslinkers. These crosslinkers are compounds having two or more ethylenically unsaturated groups, for example diacrylates or dimethacrylates of at least dihydric saturated alcohols, e.g., ethylene glycol diacrylate, ethylene glycol dimethacrylate, 1,2-propylene glycol diacrylate, 1,2-propylene glycol dimethacrylate, 1,4-butanediol diacrylate, 1,4-butanediol dimethacrylate, hexanediol diacrylate, hexanediol dimethacrylate, neopentylglycol diacrylate, neopentylglycol dimethacrylate, 3-methylpentanediol diacrylate and 3-methylpentanediol dimethacrylate. The acrylic and methacrylic esters of alcohols having more than 2 OH groups can also be used as crosslinkers, examples being trimethylolpropane triacrylate or trimethylolpropane trimethacrylate. A further class of crosslinkers comprises diacrylates or dimethacrylates of polyethylene glycols or polypropylene glycols having molecular weights of 200 to 9000 in each case. Polyethylene and/or polypropylene glycols used for preparing the diacrylates or dimethacrylates preferably have a molecular weight of 400 to 2000 each. Not only the homopolymers of ethylene oxide and/or propylene oxide can be used, but also block copolymers of ethylene oxide and propylene oxide, or random copolymers of ethylene oxide and propylene oxide, which comprise a random distribution of the ethylene oxide and propylene oxide units. Similarly, the oligomers of ethylene oxide and/or propylene oxide are useful for preparing the crosslinkers, examples being diethylene glycol diacrylate, diethylene glycol dimethacrylate, triethylene glycol diacrylate, triethylene glycol dimethacrylate, tetraethylene glycol diacrylate and/or tetraethylene glycol dimethacrylate.

Useful crosslinkers further include vinyl acrylate, vinyl methacrylate, vinyl itaconate, divinyl adipate, butanediol divinyl ether, trimethylolpropane trivinyl ether, allyl acrylate, allyl methacrylate, methylallyl methacrylate, diallyl phthalate, triallyl isocyanurate, pentaerythritol triallyl ether, triallylsucrose, pentaallylsaccharose, pentaallylsucrose, methylenebis(meth)-acrylamide, divinylethylene urea, divinylpropylene urea, divinylbenzene, divinyldioxane, triallyl cyanurate, tetraallylsilane, tetravinylsilane and bis- or polyacryloylsiloxanes (e.g., Tegomers® from Evonik Industries AG).

Crosslinkers are may be used in amounts of 0.1 to 70 wt.-%, based on the monomers to be polymerized in any one stage. Crosslinkers may be added in every stage.

It may further be advantageous to stabilize the monomer droplets and/or polymer particles with interface-active auxiliary materials. Emulsifiers or protective colloids are typically used for this purpose. Anionic, nonionic, cationic and amphoteric emulsifiers can be used. Anionic emulsifiers include, for example, alkylbenzenesulfonic acids, alkaline earth metal alkylbenzenesulfonates, sulfonated fatty acids, sulfonated olefins, sulfonated diphenyl ethers, sulfosuccinates, fatty alcohol sulfates, alkylphenol sulfates, alkyl polyglycol ether sulfates, fatty alcohol ether sulfates, fatty alcohol phosphates, alkylphenol phosphates, alkyl polyglycol ether phosphates, alkyl polyalkylene oxide phosphates, and fatty alcohol ether phosphates. Useful nonionic emulsifiers include, for example, alkylphenol ethoxylates, polysiloxane polyalkylene oxide copolymers, primary alcohol ethoxylates, fatty acid ethoxylates, alkanolamide ethoxylates, fatty amine ethoxylates, EO-PO block copolymers and alkylpolyglucosides. Useful cationic and/or amphoteric emulsifiers include for example: quaternized aminoalkoxylates, alkylbetaines, alkylamidobetaines and sulfobetaines.

Typical protective colloids include, for example, cellulose derivatives, polyethylene glycol, polypropylene glycol, copolymers of ethylene glycol and propylene glycol, polyvinyl acetate, polyvinyl alcohol, polyvinyl ether, starch and starch derivatives, dextran, polyvinylpyrrolidone, polyvinylpyridine, polyethyleneimine, polyvinylimidazole, polyvinylsuccinimide, polyvinyl-2-methylsuccinimide, polyvinyl-1,3-oxazolid-2-one, polyvinyl-2-methylimidazoline and maleic acid and/or maleic anhydride copolymers as described for example in DE 2 501 123.

Preference is given to using alkaline earth metal alkylbenzenesulfonates, alkyl polyglycol ether sulfates and polysiloxane-polyalkylene oxide copolymers.

Based on the weight of the core stage polymer, emulsifiers or protective colloids are customarily used in concentrations of 0.05 to 20 wt.-%, preferably in concentrations of 0.1 to 5 wt.-%. In the further shells, emulsifiers or protective colloids are customarily used in concentrations of 0.05 to 20 wt.-%, preferably in concentrations of 0.1 to 5 wt.-%, based on the monomers to be polymerized in this stage.

The polymerization may be carried out in a batch or continuous manner in any one of a multiplicity of versions. Customarily, some of the monomer is initially charged, optionally in a suitable diluent or solvent and optionally in the presence of an emulsifier, of a protective colloid or of further auxiliary materials, inertized and heated to the desired polymerization temperature. However, the initial charge may also merely comprise a suitable diluent. The free-radical initiator, further monomer and other auxiliary materials, e.g., chain transfer agents or crosslinkers are each optionally added in a diluent within a defined period of time. Feed times may be chosen to differ in length. For instance, a longer feed time may be chosen for the initiator feed than for the monomer feed.

When the polymer is produced in a steam-volatile solvent or solvent mixture, the solvent may be removed by introduction of steam in order that an aqueous solution or dispersion may be obtained in this way. The polymer may also be separated from the organic diluent via a drying operation.

The above described process delivers a distinctly higher scattering efficiency in coatings and hence a distinct improvement in whiteness and also particles having a distinctly larger voidage (internal water). The whiteness of the core-shell particles obtained according to the above process is ≥78. The proportion of internal water is in a range between 20% and 40%.

The following two examples are offered by way of elucidation, not limitation of the possible emulsion polymer particle to be used.

The measurement of particle size and the procedure for whiteness measurement are equal to the above described methods.

The determination of internal water content was determined as follows:

The relative internal water content, i.e., the fraction of the water population in the interior of the core shell particles based on the overall water content of the sample, can be described via a pulsed-field-gradient nuclear-magnetic resonance (PFG-NMR)¹H NMR experiment. In a system where the internal and external water populations are subject to diffusive exchange, exact determination is possible by varying the diffusion times according to Kärger (Annalen der Physik, series 7, volume 27, issue 1, 1971, pp. 107-109). A linear approximation to this exchange model is possible in the region for which the effective diffusion time A of the PFG-NMR signal attenuation is very much smaller than the exchange time between the reservoirs. In the system described, this is for example the case with A varying between 7 and 10 ms, for which the actual internal water content can be determined from the extrapolation to 0 ms. One prerequisite is that sufficiently strong gradient fields are available. In the case of exchange times being similar, a comparison of the internal water content can also be approximated via a comparison of measurements at a single, short diffusion time. In the present case, the comparisons between similar polymers were carried out with a diffusion time of Δ=7 ms by varying the gradient field strengths g up to 800 G/cm for an effective gradient pulse duration δ=1 ms by using a stimulated gradient echo pulse sequence (Steijskal & Tanner, J. Chem. Phys., 1965, Vol. 42, pp. 288ff) on a commercially available high field NMR system (Bruker Biospin, Rheinstetten/Germany). The water signal was integrated from 5.8 to 3.7 ppm relative to the water signal maximum referenced internally to 4.7 ppm. The relative signal contributions by internal and external water were derived from the prefactors of a bi-exponential fit to the gradient-dependent PFG-NMR signal drop-off, with the sum total of the two prefactors being standardized. The fitted effective diffusion coefficients in our example were on the order of 2×10−9 m²/s for external water and 5×10⁻¹² m²/s for internal water. The error associated with the determination of the internal water content was about 1% based on 100% overall water content.

Production of core-shell particles (organic raw materials not in the form of an aqueous solution were all purified by distillation prior to the synthesis):

An example for an emulsion polymer particle is as follows:

### Seed Dispersion A1

A pre-emulsion was prepared from 123.85 g of water, 0.88 g of Disponil® LDBS 20 (sodium dodecylbenzene sulfonate (20% strength)), 182 g of n-butyl acrylate, 163.45 g of methyl methacrylate and 4.55 g of methacrylic acid. The initial charge, consisting of 1172.5 g of water, 70 g of Disponil® LDBS 20 and also 22.19 g of the pre-emulsion, in a polymerization vessel equipped with an anchor stirrer, a reflux condenser and two feed vessels was heated in a nitrogen atmosphere to a temperature of 80° C. and incipiently polymerized for 15 min by addition of 67.2 g of a 2.5 wt.-% sodium peroxodisulfate solution. Thereafter, the rest of pre-emulsion was metered in at 80° C. over 60 min. This was followed by further polymerization for 15 min and cooling down to 55° C. over 20 min. To deplete the residual monomers, 3.5 g of a 10 wt.-% aqueous tert-butyl hydroperoxide solution and also 2.19 g of a 10 wt.-% aqueous Rongalit® C (sodium hydroxymethylsulfonate) solution were then added to the reaction mixture, which was stirred for one hour and then cooled down to 30° C., at which point 4.38 g of 25 wt.-% aqueous ammonia solution was added to adjust the pH of the dispersion.

Solids content: 19.8%
Particle size (PSDA, volume median): 34 nm

### Dispersion B1 (Swell-Core)

The initial charge, consisting of 1958.8 g of water and 14.54 g of seed dispersion A1, in a polymerization vessel equipped with an anchor stirrer, reflux condenser and two feed vessels was heated in a nitrogen atmosphere to a temperature of 82° C. Two minutes after addition of 26.68 g of a 7 wt.-% sodium peroxodisulfate solution, a mixture of 0.62 g of allyl methacrylate and 217.34 g of methyl methacrylate and a solution of 9.34 g of Lutensit® A-EP A (alkyl polyalkylene oxide phosphates (20% strength)), 9.34 g of Disponil® LDBS 20 and 166 g of methacrylic acid in 562 g of water were added concurrently over 90 min. Ten minutes after completion of the addition, 92.55 g of a 1.5 wt.-% sodium peroxodisulfate solution, a mixture of 62 g of n-butyl methacrylate and 345.86 g of methyl methacrylate and also a solution of 2.49 g of Disponil® LDBS 20 and 8.38 g of methacrylic acid in 276.89 g of water were added concurrently over 75 min. Finally, the feed vessel was rinsed with 33 g of water and polymerization was continued for a further 30 min.

Solids content: 21.8%
pH: 3.5
Particle size (PSDA, volume median): 186 nm

### Dispersion C1

The initial charge, consisting of 261 g of water and 273.21 g of dispersion B1, in a polymerization vessel equipped with an anchor stirrer, a reflux condenser and two feed vessels was heated in a nitrogen atmosphere to a temperature of 81° C. Addition of 25.2 g of a 1.4 wt.-% sodium peroxodisulfate solution was followed by the metered addition over 120 min of pre-emulsion 1, consisting of 132 g of water, 13.6 g of Disponil® LDBS 20, 4.08 g of methacrylic acid, 17.2 g of methyl methacrylate, 10.88 g of acrylonitrile, 3.4 g of allyl methacrylate and 202.84 g of styrene, together with 24.32 g of a 2.5 wt.-% sodium peroxodisulfate solution. On completion of the additions, 3.36 g of a 2.5 wt.-% sodium peroxodisulfate solution were added and the internal temperature was raised to 92° C. over 40 min. Then, 23.76 g of α-methylstyrene were added over 10 min and the feed rinsed with 40.5 g of water. After a further 20 min of stirring 32 g of a 10 wt.-% ammonia solution was metered in over 5 min and stirred in for 5 min. This was followed by the metered addition within 15 min of pre-emulsion 2, consisting of 98.44 g of water, 7 g of Disponil® LDBS 20, 0.28 g of methacrylic acid and 78 g of divinylbenzene (65% strength in ethylvinylbenzene). Completion of the addition was followed five minutes later by the addition of 5.64 g of a 10 wt.-% aqueous solution of tert-butyl hydroperoxide and the metering over 20 min of 31 g of a 3 wt.-% aqueous Rongalit C solution. 30 minutes after completion of the addition a further 9.16 g of a 10 wt.-% aqueous solution of tert-butyl hydroperoxide and 8.52 g of a 5.1 wt.-% aqueous Rongalit C® solution were added concurrently by metered addition over 60 min.

Solids content: 29.7%
pH: 9.5
Particle size (PSDA, volume median): 389 nm

### Whiteness: 79

Internal water: 24%

A further example for an emulsion polymer particle is as follows:

### Dispersion B2 (Swell-Core)

The initial charge, consisting of 526 g of water, in a polymerization vessel equipped with an anchor stirrer, a reflux condenser and two feed vessels was heated in a nitrogen atmosphere to a temperature of 82° C. After admixing a solution of 76 g of water, 1.41 g of Disponil® FES 993 (alkyl polyglycol ether sulfates (30% strength)) and 10.96 of EFKA® 3031 (polysiloxane polyalkylene oxide copolymers) and waiting for the temperature of the solution to return to 82° C., pre-emulsion 1 (consisting of 15.62 g of water, 0.28 g of Disponil® FES 993, 28.66 g of methyl methacrylate and 0.34 g of methacrylic acid) and 11.43 g of a 10 wt.-% sodium peroxodisulfate solution were admixed in succession before polymerizing for 30 min during which the temperature within the polymerization vessel was adjusted to 85° C. Thereafter, pre-emulsion 2 (consisting of 236 g of water, 18.63 g of Disponil® FES 993, 250 g of methyl methacrylate and 144.31 g of methacrylic acid) was metered in at 85° C. over 120 min. Finally, the feed vessel was rinsed with 10 g of water and polymerization was continued for a further 15 min.

Solids content: 33.2%
pH: 3.6
Particle size (PSDA, volume median): 130 nm

### Dispersion C2

The initial charge, consisting of 429 g of water and 80.13 g of dispersion B2 in a polymerization vessel equipped with an anchor stirrer, a reflux condenser and two feed vessels was heated in a nitrogen atmosphere to a temperature of 78° C. and, following admixture of 18 g of a 2.5 wt.-% sodium peroxodisulfate solution, incipiently polymerized for 5 min. Then pre-emulsion 1 (consisting of 30 g of water, 3 g of Disponil® LDBS 20, 2.7 g of methacrylic acid, 23.8 g of methyl methacrylate and 34 g of styrene) was added over 60 min together with 36 g of a 2.5 wt.-% sodium peroxodisulfate solution, starting at 78° C.; the internal temperature was raised to 80° C. during the addition. On completion of the additions, pre-emulsion 2 (consisting of 118 g of water, 7 g of Disponil® LDBS 20, 2 g of linseed oil fatty acids, 0.9 g of allyl methacrylate and 296.1 g of styrene) was added over 75 min together with 9 g of a 2.5 wt.-% sodium peroxodisulfate solution, starting at 80° C.; during the feed the internal temperature was raised to 82° C. On completion of the feeds the internal temperature was raised to 93° C. and the system was stirred for 15 min before 18 g of α-methylstyrene were added. After a further 40 min of stirring, the temperature was lowered to 87° C. On attaining the temperature, the system was stirred for 15 min before 228 g of a 1.7 wt.-% ammonia solution was added over 30 min. After a renewed 15 min of stirring, pre-emulsion 3 (consisting of 51 g of water, 1.2 g of Disponil LDBS 20, 0.2 g of methacrylic acid and 41.8 g of divinylbenzene) was added over 30 min. Five minutes after completion of the addition 6 g of a 10 wt.-% aqueous solution of tert-butyl hydroperoxide were admixed together with 25 g of water, while 31 g of a 3.3 wt.-% aqueous Rongalit C® solution were added over 60 min.

Solids content: 28.9%
pH: 10.2
Particle size (PSDA, volume median): 387 nm

### Whiteness: 80

Internal water: 25%

A commercially available organic white pigment in the form of a hollow emulsion polymer particle as described above is AQACeII® HIDE 6299 X from BASF.

Another suitable commercially available organic white pigment in the form of a hollow emulsion polymer particle is Ropaque™ Ultra E from Dow Chemicals.

Another suitable commercially available organic white pigment in the form of a polymer particle based on polymethyl urea resin is Pergopak® M3 from Martinswerk.

Preferably, the at least one organic white pigment is a polystyrene particle, preferably a polystyrene hollow sphere particle, or a polymethyl urea resin particle.

### Enzyme Particles

The enzyme particle according to the present invention comprises a core and a coating, wherein the core comprises at least one enzyme and the coating comprises at least one organic white pigment.

Within the meaning of the invention, it is to be understood that the enzyme particle (especially the coating of the enzyme particle) may comprise additional organic white pigments. Thus, in one embodiment, the coating of the enzyme particle comprises a mixture of at least two different organic white pigments.

Within the meaning of the present invention, the terms “enzyme containing particle” and “enzyme particle” are interchangeable. The enzyme particle according to the present invention is a small particle containing at least one enzyme and an organic white pigment. The enzyme particle may be shaped spherical. The person skilled in the art will however know, that the enzyme particles are not limited to strict spherical shapes but that they may also have the form of e.g. an ellipsoid. The enzyme particle may further be shaped spherical or ellipsoid having an uneven surface. According to the present invention, the enzyme particle is in the form of an enzyme granule.

Within the meaning of the invention, it is to be understood that a small particle, such as the enzyme particle, typically has a diameter of 20 to 2000 μm, preferably 50 to 1500 μm, more preferably 250 to 1200 μm.

In one embodiment, the enzyme particle does not include a surfactant, a detergent builder, and/or a bleaching agent. In another embodiment, the enzyme particle includes less than 10 wt.-%, or less than 5 wt.-%, or less than 2 wt.-%, or less than 1 wt.-% surfactant. In a particular embodiment, the surfactant is a laundry detergent surfactant.

In a certain embodiment, the coating of the enzyme particle contains less than 35 wt.-%, preferably less than 15 wt.-%, preferably less than 10 wt.-%, preferably less than 5 wt.-%, more preferably less than 1 wt.-%, more preferably less than 0.5 wt.-%, and even more preferably less than 0.1 wt.-% inorganic white pigment, preferably none inorganic white pigment.

In a preferred embodiment, the coating of the enzyme particle contains less than 10 wt.-%, preferably less than 5 wt.-%, more preferably less than 1 wt.-%, more preferably less than 0.5 wt. %, and even more preferably less than 0.1 wt.-% titanium dioxide. In a particular embodiment, the coating of the enzyme particle does not comprise titanium dioxide.

In a preferred embodiment, the enzyme particle contains less than 1 wt.-%, preferably less than 0.5 wt.-%, more preferably less than 0.1 wt.-%, more preferably less than 0.05 wt.-%, and even more preferably less than 0.01 wt.-% titanium dioxide. In a particular embodiment, the enzyme particle does not comprise titanium dioxide.

Within the meaning of this invention, the core of the enzyme particle comprises a granule, a sugar crystal, a salt crystal, and a non-pareil. Granules may exemplarily be in the form of marums, layered granules, prills, drum granules, or agglomerated granules. The core may also comprise a layer structure, wherein the enzyme is comprised in at least one of the layers. The core may also consist of an inert particle with the blend absorbed into it, or with the blend applied onto the surface, e.g., via fluid bed coating.

The core of the enzyme particle comprises the enzyme and at least one additional ingredient. Additional ingredients may be selected from the group consisting of acidic buffer components, antioxidants, binders such as synthetic polymer, wax, fat, or carbohydrate, fillers, fibre materials (e.g. cellulose or synthetic fibres), fragrances, light spheres, lubricants, peroxide decomposing catalysts, plasticizers, salts, salts of multivalent cations, reducing agents, solubilizing agents, stabilizing agents, suspension agents, and/or viscosity regulating agents.

The core of the enzyme particle may have a diameter of at least 20 μm. Preferably, the core of the enzyme particle may have a diameter of 20-2000 μm, preferably 50-1500 μm, 100-1500 μm or 250-1200 μm.

The core can be prepared by granulating a blend of the ingredients, e.g., by a method comprising granulation techniques such as crystallization, marumerization, precipitation, pan-coating, fluid bed coating, fluid bed agglomeration, spray granulation, rotary atomization, extrusion, prilling, spheronization, size reduction methods, drum granulation, high shear granulation, or other suitable processes.

Methods for preparing the core can be found in Handbook of Powder Technology; Particle size enlargement by C. E. Capes; Volume 1; 1980; Elsevier. Preparation methods include known feed and granule formulation technologies, e.g.:
a) Spray dried products, wherein a liquid enzyme containing solution is atomized in a spray drying tower to form small droplets which during their way down the drying tower dry to form an enzyme containing particle material. Very small particles can be produced this way (Michael S. Showell (editor); Powdered detergents; Surfactant Science Series; 1998; vol. 71; page 140-142; Marcel Dekker).
b) Layered products, wherein the enzyme is coated as a layer around a pre-formed inert core particle, wherein an enzyme containing solution is atomized, typically in a fluid bed apparatus wherein the pre-formed core particles are fluidized, and the enzyme containing solution adheres to the core particles and dries up to leave a layer of dry enzyme on the surface of the core particle. Particles of a desired size can be obtained this way if a useful core particle of the desired size can be found. This type of product is described in, e.g., WO 97/23606. It is also possible to apply this layering technology on enzyme containing particles as starting seeds. When the seed particles are produced out of the layering liquid itself, e.g. by spray drying in fluid bed, homogenuous particles are obtained. Spray granulation technology is suitable to obtain such particles (H. Uhlemann, L. Mörl, Wirbelschicht-Spruhgranulation, Springer-Verlag Berlin, 2000). If spray-granulation is used, the enzyme is homogenuously distributed in the particle.
c) Absorbed core particles, wherein rather than coating the enzyme as a layer around the core, the enzyme is absorbed onto and/or into the surface of the core. Such a process is described in WO 97/391 16.
d) Extrusion or pelletized products, wherein an enzyme containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme (see also Michael S. Showell (editor); Powdered detergents; Surfactant Science Series; 1998; vol. 71; page 140-142; Marcel Dekker).
e) Prilled products, wherein an enzyme containing powder is suspended in molten wax and the suspension is sprayed, e.g., through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. Also U.S. Pat. Nos. 4,016,040 and 4,713,245 are documents relating to this technique.
f) Mixer granulation products, wherein a liquid is added to a dry powder composition of, e.g., conventional granulating components, the enzyme being introduced either via the liquid or the powder or both. The liquid and the powder are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process wherein various high-shear mixers can be used as granulators, granulates consisting of enzyme as enzyme, fillers and binders etc. are mixed with cellulose fibers to reinforce the particles to give the so-called T-granulate. Reinforced particles, being more robust, release less enzymatic dust.
g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons).
h) Fluid bed granulation. Fluid bed granulation involves suspending particles in an air stream and spraying a liquid onto the fluidized particles via nozzles. Particles hit by spray droplets get wetted and become tacky. The tacky particles collide with other particles and adhere to them and form a granule.
i) The cores may be subjected to drying, such as in a fluid bed drier. Other known methods for drying granules in the feed or detergent industry can be used by the skilled person. The drying preferably takes place at a product temperature of from 25 to 90° C. For some enzymes it is important the cores comprising the enzyme contain a low amount of water before coating. If water sensitive enzymes are coated before excessive water is removed, it will be trapped within the core and it may affect the activity of the enzyme negatively. After drying, the cores preferably contain 0.1-10% wt.-% water.

Typically, the enzyme granules are sifted after drying to remove the overs and the fines which may be recycled.

The enzyme particle according to the present invention further comprises a coating, covering the surface of the core. The coating comprises the organic white pigment. The organic white pigment can additionally be present or absent from the core. Preferably, the core does not comprise the organic white pigment.

Within the meaning of the invention, the coating forms a continuous coating covering the entire surface of the core or may only partially cover the surface of the core. In case the coating only partially covers the surface of the core, the coating preferably covers at least 30%, preferably at least 50%, more preferably at least 70%, in particular at least 90%, of the surface of the core.

In a certain embodiment, the coating contains less than 3 wt.-%, preferably less than 0.1 wt.-%, inorganic white pigment.

In the coating composition of the enzyme particle, the currently employed pigments, specifically TiO₂, can be wholly or partly replaced by the organic white pigment, which may be applied as dispersion, as described herein.

In one embodiment, the coating is from 5 to 20 wt.-%, of the total weight of the enzyme particle. In one embodiment, the coating of the enzyme particle comprises the at least one organic white pigment as described herein in the range of from 10 to 90 wt.-%, based on the total weight of the coating, in one embodiment, 30 to 70 wt.-%, and optionally a binder.

Any known suitable binders for coatings, such as exemplarily polyethylene glycol (PEG, e.g., PEG 9000, PEG 12000), methyl hydroxy-propyl cellulose (MHPC), polyvinylpyrrolidone (PVP, e.g., Luvitec VA64 from BASF) and polyvinyl alcohol (PVA, e.g., Mowiol 3-85, from Kuraray), may be used.

In one embodiment, the coating is at least 0.1 μm thick, preferably at least 0.5 μm, at least 1 μm or at least 5 μm. In a certain embodiment, the thickness of the coating is below 100 μm, preferably below 60 μm, and more preferably below 40 μm.

In another embodiment, the thickness of the coating is from 0.1 to 100 μm, preferably from 0.5 to 40 μm, or from 1 to 20 μm, or from 5 to 20 μm.

In particular for a detergent particle the thickness of the coating is below 40 μm, preferably below 20 μm, and more preferably below 10 μm. In another embodiment, the thickness of the coating of a detergent particle is from 0.1 to 40 μm, or from 0.1 to 20 μm, or from 1 to 15 μm. The coating can further contain other materials as known in the art, e.g., fillers, antisticking agents, pigments, dyes, plasticizers, additional filming polymers, further auxiliaries, and/or binders. It is preferred that the coating does not comprise titanium dioxide.

According to a certain embodiment, the coating may include additional pigments, for example, inorganic white pigments such as titanium dioxide, preferably in the rutile form, barium sulfate, zinc oxide, zinc sulfide, basic lead carbonate, antimony trioxide, lithopone (zinc sulfide and barium sulfate) or colored pigments, for example iron oxides, carbon black, graphite, zinc yellow, zinc green, ultramarine, manganese black, antimony black, manganese violet, Prussian blue or Parisian green. In one preferred embodiment, no further inorganic white pigment is comprised in the coating. In particular, no titanium dioxide is comprised in the coating.

Customary auxiliaries include wetting or dispersing agents, such as sodium polyphosphate, potassium polyphosphate, ammonium polyphosphate, alkali metal and ammonium salts of acrylic acid copolymers or of maleic anhydride copolymers, polyphosphonates, such as sodium 1-hydroxyethane-1,1-diphosphonate and also naphthalenesulfonic acid salts, in particular their sodium salts.

The coating may be carried out by various technologies, e.g. mixer coating or coating in a fluidized bed. The coating material can be applied to the core, such as granules, in a molten state, or out of a solution.

The whiteness of enzyme particles may be assessed by an equal photometric device as described above which measures the colorimetric L*a*b-values of a collection of granules. Essentially, the light reflected from the granules is measured and transferred to absolute colorimetric values using calibration standards. The higher the L-value, the higher the whiteness of the enzyme particles. As the L-value of the enzyme particles further depends on the corresponding coated core, different coating compositions must be applied to the same core material in order to allow conclusions on whitening.

The abrasion stability of the enzyme particles may be assessed in test protocols like e.g. the Heubach test where a sample is mechanically stressed and the generated dust is being collected and analysed. Higher abrasion stability of the enzyme particle is reflected in lower dust level and most specifically in lower enzyme content of the dust.

Abrasion stability of the enzyme particle may e.g. be determined with a Heubach Dustmeter Type Ill. In this configuration, the enzyme particles are subjected to the action of four moving steel balls in a cylindrical container. This mechanical action generates dust which is separated from the enzyme particles by a constant air flow through the sample container, and is then collected in a microfilter.

25 ml of enzyme particles are used in the Heubach test. Enzyme particle samples are sieved to 500-1250 μm prior to testing. Measurement settings are 45 rpm rotor speed, air flow 20 L/min and 20 minutes test duration. The total amount of dust is obtained by weighting the filter before and after the test. The enzyme level contained in the dust is determined by a standard protease enzyme assay. The result of the Heubach test is the total mass of enzyme in the dust, normalized to the weight of the enzyme particle sample. Lower values mean a lower risk in enzyme dust generation during handling of the enzyme particles.

The present invention further relates to the use of at least one organic white pigment in the coating of enzyme particles. The organic white pigment is preferably an organic white pigment as described above.

In one embodiment, the at least one organic white pigment is used to improve the whiteness of enzyme particles. The improved whiteness refers to an improvement in comparison to an enzyme particle not including a whitening agent. Under improved whiteness, it is also to be understood that the enzyme particles, comprising an organic white pigment as described herein, exhibit an L-value which is at least as high as the L-value of enzyme particles, comprising a whitening agent different to the organic white pigments as described herein. Preferably, the L-value of the enzyme particles, comprising an organic white pigment as described herein, is higher, compared to the enzyme particle, comprising a whitening agent different to the organic white pigments as described herein. Preferably, the L-value of the enzyme particles, comprising an organic white pigment as described herein, is higher, compared to the enzyme particle, comprising TiO2 as whitening agent. The L-value is measured as described above for the organic white pigment.

In one embodiment, the enzyme particle according to the invention possesses an L-value, determined as described above, of at least 70, preferably of at least 75 or of at least 76. In another embodiment, the enzyme particle according to the invention possesses an L-value, determined as described above, of from 70 to 95, preferably of from 75 to 85.

In another embodiment, the at least one organic white pigment is used to increase the abrasion resistance of enzyme particles. Again, the increase of the abrasion resistance is to be seen in relation to untreated enzyme particles, or to enzyme particles comprising a whitening agent different to the organic white pigments as described herein. Under increase of the abrasion resistance it is to be understood that the enzyme particle, comprising an organic white pigment as described herein, exhibits a lower dust level as an enzyme particle, comprising a whitening agent different to the organic white pigments (preferably comprising TiO2 as whitening agent) as described herein, determined via e.g., a Heubach test as described above in more detail.

In one embodiment, the enzyme particle according to the invention possesses less than 0.6, preferably less than 0.5 μg enzyme dust/g, determined as described above. In another embodiment, the enzyme particle according to the invention possesses about 0.001 to 0.6 μg enzyme dust/g, preferably 0.01 to 0.5 μg enzyme dust/g, determined as described above.

In one embodiment, the at least one organic white pigment is used to improve the whiteness and to increase the abrasion resistance of enzyme particles.

In a particular embodiment of the above described uses, the at least one organic white pigment is in the form of hollow organic particles.

### Enzyme

The core of the enzyme particle according to the invention comprises at least one enzyme in an amount of from 0.1 wt.-% to 20 wt.-%, preferably from 0.5 wt.-% to 15 wt.-%, more preferably from 1 wt.-% to 10 wt.-%, in particular from 2 wt.-% to 8 wt.-%, based on the total weight of the core.

In one aspect of the present invention, the enzyme of interest is a detergent enzyme.

In another aspect of the present invention, the enzyme of interest is a food and/or feed enzyme, which may be included in animal feed or food compositions, e.g., pet or livestock food.

It is to be understood that the enzymes used are active enzyme proteins, ribozymes, or deoxyribozymes.

Enzymes of interest are in particular enzymes classified as oxidoreductase (EC 1), transferase (EC 2), hydrolase (EC 3), lyase (EC 4), isomerase (EC 5), or ligase (EC 6) (EC-numbering according to Enzyme Nomenclature, Recommendations (1992) of the Nomenclature Committee of the International Union of Biochemistry and Molecular Biology including its supplements published 1993-1999).

Oxidoreductases that may be considered according to the invention include peroxidases, and oxidases such as laccases.

An enzymes exhibiting peroxidase activity may be any peroxidase enzyme comprised by the enzyme classification (EC 1.11.1.7), or any fragment derived therefrom, exhibiting peroxidase activity.

Particularly, a recombinantly produced peroxidase is preferred, e.g., a peroxidase derived from a Coprinus sp., in particular C. macrorhizus or C. cinereus according to WO 92/16634, or a variant thereof, e.g., a variant as described in WO 93/24618 and WO 95/10602.

In the context of this invention, laccases and laccase related enzymes contemplate any laccase enzyme comprised by the enzyme classification (EC 1.10.3.2), any catechol oxidase enzyme comprised by the enzyme classification (EC 1.10.3.1), any bilirubin oxidase enzyme comprised by the enzyme classification (EC 1.3.3.5) or any monophenol monooxygenase enzyme comprised by the enzyme classification (EC 1.14.18.1).

The microbial laccase enzyme may be derived from bacteria or fungi (including filamentous fungi and yeasts) and suitable examples include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinus, e.g. C. plicatiis and C. cinereus, Psatyrella, Myceliophthora, e.g., M. thermophila, Schytalidium, Polyporus, e.g., P. pinsitus, Phlebia, e.g., P. radita (WO 92/01046), or Coriolus, e.g., C. hirsutus (JP 2-238885), in particular laccases obtainable from Trametes, Myceliophthora, Schytalidium, or Polyporus.

In one embodiment, the enzyme according to the invention is a hydrolase (EC 3), e.g., a glycosidase (EC 3.2), or a peptidase (EC 3.4). Preferred enzymes are enzymes selected from the group consisting of an amylase (e.g., an alpha-amylase (EC 3.2.1.1), a cellulase (EC 3.2.1.4), a lactase (EC 3.2.1.108), a lipase, and a protease; in particular an enzyme selected from the group consisting of amylase, protease, lipase, and cellulase, preferably, amylase or protease.

In a certain embodiment, the following hydrolases are preferred:

Suitable proteases include those of bacterial or fungal origin. The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Preferably, the subtilisin protease is a serine protease that uses a catalytic triad composed of Asp32, His64 and Ser221 (subtilisin BPN′ numbering), preferably, the pH value of the subtilisin protease is between pH 7.0 and pH 10.0, preferably between pH 8.0 and pH 9.5. Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274.

Additional useful proteases are described in WO2012080201 and WO2013060621.

A further preferred protease is a protease according to SEQ ID NO: 1 of DE102012215642A1 and variants thereof, wherein the preferred variants comprises one or more mutations at position 3, 4, 99, 194 and 199 (using the numbering of the alkaline protease from DSM5483), preferably comprising one or more of the following mutations: S3T, V41, R99E, V194M, and V1991, preferably, S3T, V41, R99E, and V1991, more preferably R99E, or R99E in combination with two additional mutations selected from the group consisting of S3T, V41, and V1991, preferably SEQ ID NO: 1 of DE102012215642A1 with R99E, or S3T, V41, V194M, and V1991, or S3T, V41 and V1991. A further preferred protease is a protease according to SEQ ID NO: 2 of DE102012215642A1 and variants thereof, wherein the preferred variants comprises a mutation at position 99 and an insertion between position 99 and 100, wherein the insertion is an aspartate (Asp, D) residue. In this embodiment, preferably the mutation at position 99 is S99A. Further preferred protease variants are SEQ ID NO: 7 of DE102011118032A1 comprising the mutations S3T, V41 and V2051 or SEQ ID NO: 8 of DE102011118032A1 comprising the mutations S3T, V41, V193M, V1991, and L211D using the numbering of the alkaline protease from DSM5483.

Preferred commercially available protease enzymes include Alcalase™, Savinase™, Primase™, Duralase™, Esperase™, and Kannase™ (Novozymes A/S), Maxatase™, Maxacal™, Maxapem™, Properase™, Purafect™, Purafect OxP™, FN2™, and FN3™ (Genencor International Inc.).

Suitable lipases include those of bacterial or fungal origin. Examples of useful lipases include lipases from Humicola (synonym Thermomyces), e.g., from H. lanuginosa (T. lanuginosus), as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al. (1993), Biochemica et Biophysica Acta, 1131, 253-360), B. stearothermophilus (JP 64/744992), or B. pumilus (WO 91/16422).

Other examples of lipases are e.g. phospholipases, such as the mammalian pancreatic phospholipases A2. Further examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079, and WO 97/07202.

Preferred commercially available lipase enzymes include Lipolase™ and Lipolase Ultra™ (Novozymes A/S).

Suitable amylases (alpha and/or beta) include those of bacterial or fungal origin. Chemically modified or protein engineered mutants are included. Amylases include, for example, alpha-amylases obtained from Bacillus, e.g., a special strain of B. licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Duramyl™, Termamyl™, Fungamyl™ and BANT™ (Novozymes A/S), Rapidase™ and Purastar™ (from Genencor International Inc.).

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and PCT/DK98/00299.

Further suitable cellulases are plant cell wall degrading enzymes of which include cellulases such as R-glucanases, hemicellulases such as xylanases, or galactanases.

Commercially available cellulases include Celluzyme™, and Carezyme™ (Novozymes A/S), Clazinase™, and Puradax HA™ (Genencor International Inc.), and KAC-500 (B)™ (Kao Corporation).

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619 or WO 2011/085747. A commercially available mannanase is Mannaway (Novozymes A/S).

Suitable phosphatases include phytases (e.g., 3-phytases and 6-phytases) and/or acid phosphatases. Suitable Phytases are decribed in WO 91/05053, WO 2011/048046 and WO 2012/143862. Further suitable enzymes include but are not limited to carbohydrases, such as amylolytic enzymes, galactosidases, pectinases, and esterases.

The lyase may be a pectate lyase of bacterial or fungal origin. Chemically or genetically modified mutants are included. In one embodiment, the pectate lyase is derived from Bacillus, preferably Bacillus substilis, B. licherniformis or B. agaradhaerens, or a variant derived of any of these, e.g., as described in U.S. Pat. No. 6,124,127, WO 1999/027083, WO 1999/027084, WO 2002/006442, WO 2002/092741, WO 2003/095638, commercially available pectate lyases include XPect, Pectawash and Pectaway (Novozymes A/S).

Further, protein engineered variants of a protein of interest, made by recombinant DNA techniques or by chemical modification, may be of particular interest.

### Washing or Cleaning Composition

The invention further relates to a washing or cleaning composition, comprising enzyme particles according to the present invention.

In one embodiment, the invention relates to a washing or cleaning composition, comprising enzyme particles according to the present invention and a bleach.

The washing or cleaning composition of the present invention may be formulated, for example, as a hand or machine washing or cleaning composition. For laundry washing or cleaning compositions a laundry additive composition may be included, suitable for pre-treatment of stained fabrics and a rinse added fabric softener composition. The washing or cleaning composition may also be formulated as a washing or cleaning composition for use in general household hard surface cleaning operations, or be formulated for hand or machine dishwashing operations.

The washing or cleaning composition comprising enzyme particles of the present invention can be a liquid composition or a powder composition. Preferably, the washing or cleaning composition comprising enzyme particles of the present invention is a powder composition.

In one embodiment, the invention is directed to washing or cleaning compositions comprising enzyme particles of the present invention in combination with one or more additional washing or cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment, of the present invention, the enzyme particles may be added to a washing or cleaning composition in an amount corresponding to 0.001-200 mg of enzyme, such as 0.005-100 mg of enzyme, preferably 0.01-50 mg of enzyme, more preferably 0.05-20 mg of enzyme, even more preferably 0.1-10 mg of enzyme per liter of wash liquor or of wash powder.

The washing or cleaning composition may comprise one or more surfactants, which may be anionic and/or cationic and/or non-ionic and/or semi-polar and/or zwitterionic, or mixtures thereof.

Surfactants are typically present in the washing or cleaning composition in a level of from about 0 to about 60 wt.-%, preferably from about 1 to about 40 wt.-%, or from about 3 to about 20 wt.-%. Surfactants are chosen based on the desired cleaning application, and include any conventional surfactant known in the art. Any surfactant known in the art for use in detergents may be utilized. In one embodiment, the washing or cleaning composition does not comprise a surfactant.

In one embodiment, the washing or cleaning composition comprises at least one anionic surfactant, such as for example, a sulfate, sulfonate, or carboxylate surfactant, or a mixture thereof. Preferred sulfates are those having from 12 to 22 carbon atoms in the alkyl radical, optionally in combination with alkylethoxysulfates having from 10 to 20 carbon atoms in the alkyl radical.

Preferred sulfonates are, for example, alkylbenzenesulfonates having from 9 to 15 carbon atoms in the alkyl radical. The cation in the anionic surfactants is preferably an alkali metal cation, especially sodium.

Preferred carboxylates are alkali metal sarcosinates of formula Rₐ—CO—N(R_{b})—CH₂COOM′₁, wherein

Rₐ is alkyl or alkenyl having from 8 to 18 carbon atoms in the alkyl or alkenyl radical,
R_{b} is C₁-C₄alkyl, and
M′₁ is an alkali metal.

In one embodiment, the washing or cleaning composition comprises cationic surfactants, such as for example, alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

In one embodiment, the washing or cleaning composition comprises at least one non-ionic surfactant, such as for example, a primary or secondary alcohol ethoxylate, especially a C₈-C₂₀ aliphatic alcohol ethoxylated with an average of from 1 to 20 mol of ethylene oxide per alcohol group.

Preference is given to primary and secondary C₁₀-C₁₅ aliphatic alcohols ethoxylated with an average of from 1 to 10 mol of ethylene oxide per alcohol group.

Non-ethoxylated non-ionic surfactants, for example alkylpolyglycosides, glycerol monoethers and polyhydroxyamides (glucamide), may likewise be used.

In one embodiment, the washing or cleaning composition comprises at least one semi-polar surfactant, such as for example, amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

In one embodiment, the washing or cleaning composition comprises at least one zwitterionic surfactant, such as for example, betaine, alkyldimethylbetaine, sulfobetaine, and combinations thereof.

The washing or cleaning composition may comprise at least one builder and/or co-builder in an amount of from about 0 to about 65 wt.-%, or from 5 to 50 wt.-%, depending on the application of the final product. Suitable builders are considered to be, for example, alkali metal phosphates, especially tripolyphosphates, carbonates and hydrogen carbonates, especially their sodium salts, silicates, aluminum silicates, polycarboxylates, polycarboxylic acids, organic phosphonates, aminoalkylenepoly(alkylenephosphonate(s)), and mixtures of such compounds.

Silicates that are especially suitable are sodium salts of crystalline layered silicates of the formula NaHSitO(2t+1). pH2 or Na2SitO(2t+1) pH2O wherein t is a number from 1.9 to 4 and p is a number from 0 to 20.

Among the aluminum silicates, preference is given to those commercially available under the names zeolite A, B, X and HS, and also to mixtures comprising two or more such components. Among the polycarboxylates, preference is given to polyhydroxycarboxylates, especially citrates, and acrylates, and also to copolymers thereof with maleic anhydride. Preferred polycarboxylic acids are nitrilotriacetic acid (NTA), ethylenediaminetetraacetic acid (EDTA) and ethylenediamine disuccinate either in racemic form, or in the enantiomerically pure (S,S) form. Phosphonates or aminoalkylenepoly(alkylenephosphonate(s)) that are especially suitable are alkali metal salts of 1-hydroxyethane-1,1-diphosphonic acid, nitrilotris(methylenephosphonic acid), ethylenediaminetetramethylenephosphonic acid, and diethylenetriaminepentamethylenephosphonic acid.

The washing or cleaning composition may comprise at least one bleaching system known in the art in an amount of from 0 to 50 wt.-%. Suitable bleaching components include bleaching catalysts, photobleaches, bleach activators, sources of hydrogen peroxide such as sodium percarbonate and sodium perborates, preformed peracids, and mixtures thereof.

Suitable peroxide components are for example, the organic and inorganic peroxides known in the literature and obtainable commercially that bleach textile materials at conventional washing temperatures, for example at from 10 to 95° C.

Suitable organic peroxides are, for example, mono- or poly-peroxides, especially organic peracids or salts thereof, such as phthalimidoperoxycaproic acid, peroxybenzoic acid, diperoxydodecanoic diacid, diperoxynonanoic diacid, diperoxydecanoic diacid, diperoxyphthalic acid, or salts thereof.

Suitable bleach activators are, for example, polyacylated alkylenediamines, especially tetraacetylethylenediamine (TAED), acylated glycolurils, especially tetraacetylglycoluril (TAGU), N,N-diacetyl-N,N10 dimethylurea (DDU), and acylated triazine derivatives, especially 1,5-diacetyl-2,4-dioxohexahydro-1,3,5-triazine (DADHT).

The peroxides are added to the composition preferably by mixing the components, for example using a screw metering system and/or a fluidized bed mixer.

The washing or cleaning composition may comprise at least one hydrotrope known in the art in an amount of from 0 to 5 wt.-%. Hydrotropes are classically used across industries from pharma, personal care, food, to technical applications. Use of hydrotropes in washing or cleaning compositions allow for example more concentrated formulations of surfactants (as in the process of compacting liquid detergents by removing water) without inducing undesired phenomena such as phase separation or high viscosity. Suitable hydrotropes are, for example, sodium benzene sulfonate, sodium p-toluene sulfonate (STS), sodium xylene sulfonate (SXS), sodium cumene sulfonate (SCS), sodium cymene sulfonate, amine oxides, alcohols and poly-glycolethers, sodium hydroxynaphthoate, sodium hydroxynaphthalene sulfonate, sodium ethylhexyl sulfate, and combinations thereof.

The washing or cleaning compositions may furthermore comprise dirt-suspending agents, for example sodium carboxymethylcellulose; pH regulators, for example alkali metal or alkaline earth metal silicates; bactericides; foam regulators, for example soap; salts for adjusting the spray drying and the granulating properties, for example sodium sulfate; fragrances; antistatic agents; fabric conditioners; further bleaching agents; pigments; and/or toning agents.

In view of the above, the invention further relates to the use of enzyme particles according to the invention, optionally together with a detergent compound for washing or cleaning stains or soiling on e.g. textile material or surfaces in the context of a washing or a cleaning process. The washing or cleaning process may be at a temperature between 10 and 95° C., preferably between 20 and 60° C. The washing or cleaning process is preferably carried out in an automatic washing machine.

### Food or Feed Composition

In another certain embodiment, the invention relates to a food or feed composition, comprising enzyme particles according to the present invention.

For the preparation of a food or feed composition, or a premix or a precursor suitable for animal nutrition, the process may comprise the mixing a stabilized solid and/or liquid formulation comprising enzyme particles according to the present invention with one or more food substance(s) or ingredient(s).

Suitable stabilizing agents may be selected from the group consisting of gummi arabicum, at least one plant protein and mixtures thereof. It is understood that the stabilizing agent can be selected from one agent, e.g. only gummi arabicum or be composed of a mixture of e.g. one plant protein and gummi arabicum or a mixture of two or three or more different plant proteins. In one embodiment, the stabilizing agent is gummi arabicum. In another embodiment, the stabilizing agent is at least one plant protein.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

In the following (unlimiting) examples, the enzyme granules were coated with an aqueous coating mixture in a fluidized bed. The expert skilled in the art will know how to transfer the teachings of this application to a different coating technology.

### Example 1

75 g of Pluriol E 9000 (PEG 9000, BASF) and 250 g of AQACell 6299 X (Polystyrene particles in water, 30 wt.-% solids, BASF) were blended with 125 g of cold water by continuous stirring to obtain a homogenuous dispersion. The solid content of the coating dispersion was 33.3 wt.-%. 1080 g of enzyme core granules containing 60 wt.-% of ammonium sulfate (from BASF), 30 wt. % of china clay (from Sigma-Aldrich), 4 wt.-% solids of a polyacrylate sodium salt (Sokalan Pa. 25, from BASF), 5 wt.-% of protease and 1 wt.-% of water were introduced into a laboratory fluid bed (Glatt Procell 5) equipped with a bottom spray setup. After fluidization of the enzyme core granules, 310 g of the coating solution was sprayed onto the pellets within 31 minutes. Inlet air temperature was 56° C., and product temperature was about 40° C. When spraying of the coating solution was finished, the heating of the inlet air was turned off, and the enzyme granules were cooled until the product temperature was about 30° C. After discharging, 1080 g of enzyme granules were obtained.

The coating level of the final enzyme granule was 9.2 wt.-%. Residual moisture was 1.5 wt.-%. The total level of active enzyme was 4.5 wt.-%.

### Reference Example 2

75 g of Pluriol E 9000 (PEG 9000, BASF) and 75 g of TiO₂ (TiO₂, Sachtleben) were blended with 300 g of cold water by continuous stirring to obtain a homogenous dispersion. The solid content of the coating dispersion was 33.3 wt.-%.

1030 g of the same enzyme core granules as in experiment Ex. 1 were introduced into a laboratory fluid bed (Glatt Procell 5) equipped with a bottom spray setup. After fluidization of the enzyme core granules, 310 g of the coating solution was sprayed onto the enzyme core granules within 26 minutes. Inlet air temperature was 54° C., and product temperature was about 40° C. When spraying of the coating solution was finished, the heating of the inlet air was turned off, and the enzyme granules were cooled until the product temperature was about 30° C. The product was discharged and 1040 g of enzyme granules were obtained.

The coating level of the final enzyme granule was 9.1 wt.-%. Residual moisture was 1.2 wt.-%. The total level of active enzyme was 4.5 wt.-%.

### Reference Example 3-4

Using the same enzyme core granules and the same coating procedure as outlined in examples Ex. 1 and Ref. 2, further enzyme granules Ref. 3 and 4 were being produced.

Coating materials were the same like in experiments Ex. 1 and Ref. 2-4. Further binder materials were PEG 12000 (from BASF) and PVA (Mowiol 3-85, from Kuraray, which is a partially saponified polyvinylalcohol).

In all experiments, 10 parts of solids of the coating composition were sprayed on 100 parts of enzyme core granules. The final coating level in the coated enzyme particles was about 9 wt.-%.

Table 1 gives a summary of the coating compositions, based on wt.-% solids, of Ex. 1, Ref. 2-4.

| Sample No. | Ex. 1 | Ref. 2 | Ref. 3 | Ref. 4 |
|---|---|---|---|---|
| TiO₂ | | 50 | 50 | 50 |
| Polystyrene particles | 50 | | | |
| PEG 9000 | 50 | 50 | | 25 |
| PEG 12000 | | | 50 | |
| PVA (Mowiol 3-85, from Kuraray) | | | | 25 |

The coated enzyme granules of the examples have a d50 value of about 520 μm, as measured by Camsizer, wherein the measurement is based on dynamic image analysis.

### (Reference) Example 5-11

For these coating experiments, enzyme cores containing 54 wt.-% of ammonium sulfate (from BASF), 30 wt.-% of china clay (from Sigma-Aldrich), 4 wt.-% solids of a polyacrylate sodium salt (Sokalan Pa. 25, from BASF), 5 wt.-% of cellulosic fibres (Arbocel FD600/30), 5 wt.-% of protease and about 2 wt.-% of water were used.

Coating materials were the same like in experiments Ex. 1 and Ref. 2-4. Further white pigments were zeolithe ZP-4A (from Silkem), talkum TP-1 (from Scheruhn) and Pergopak M (polymethyl urea resin, from Martinswerk). The coating was applied in the same procedure as outlined in examples Ex. 1 and Ref. 2.

The final coating level in the coated enzyme particles was about 9 wt.-%.

Table 2 gives a summary of the coating compositions, based on wt.-% solids, of Ex. 5-8, Ref. 9-11.

| | Sample No. | | | | | | |
|---|---|---|---|---|---|---|---|
| | Ex. | Ex. | Ex. | Ex. | Ref. | Ref. | Ref. |
| | 5 | 6 | 7 | 8 | 9 | 10 | 11 |
| TiO₂ | | 25 | | | 50 | | |
| Zeolithe | | | | 25 | | 50 | |
| Talkum | | | | | | | 75 |
| Polystyrene particles | 67 | | | | | | |
| Pergopak M | | 25 | 75 | 25 | | | |
| PEG 9000 | 33 | 50 | 25 | 50 | 50 | 50 | 25 |

### (Reference) Example 12-20

For these coating experiments, enzyme cores containing 31 wt.-% of ammonium sulfate (from BASF), 59 wt.-% of china clay (from Sigma-Aldrich), 4 wt.-% solids of a polyacrylate sodium salt (Sokalan Pa. 25, from BASF), 5 wt.-% of protease and about 1 wt.-% of water were used.

Coating materials were the same like in (reference) experiments 1-11. A further binder material was Luvitec VA64 (a vinylpyrrolidone-vinylacetate copolymer, from BASF). The coating was applied in the same procedure as outlined in examples Ex. 1 and Ref. 2. The final coating level in the coated enzyme particles was about 9 wt.-%.

Table 3 gives a summary of the coating compositions, based on wt.-% solids, of Ex. 12-15, Ref. 16-20.

| | Sample No. | | | | | | | | |
|---|---|---|---|---|---|---|---|---|---|
| | Ex. | Ex. | Ex. | Ex. | Ref. | Ref. | Ref. | Ref. | Ref. |
| | 12 | 13 | 14 | 15 | 16 | 17 | 18 | 19 | 20 |
| TiO₂ | | | | | 50 | 67 | 67 | 50 | 67 |
| Zeolithe | | | 25 | | | | | | |
| Polystyrene | 67 | 67 | | 50 | | | | | |
| particles | | | | | | | | | |
| Pergopak M | | | 25 | | | | | | |
| PEG 9000 | | 16.5 | 50 | 25 | 50 | | | 25 | 33 |
| PVA | 33 | 16.5 | | 25 | | 33 | | 25 | |
| Luvitec VA64 | | | | | | | 33 | | |
| (BASF) | | | | | | | | | |

### (Reference) Example 21-22

The enzyme cores contained 59 wt.-% of magnesium sulfate (from BASF), 34 wt.-% of china clay (from Sigma-Aldrich), 1 wt.-% solids of a polyacrylate sodium salt (Sokalan Pa. 25, from BASF), 5 wt.-% of protease and about 1 wt.-% of water.

Coating materials and final coating level on the enzyme cores was the same as outlined in the previous examples. The solid content of the coating slurry was set to 15 wt.-% in both trials.

Table 4 gives a summary of the coating compositions, based on wt.-% solids, of Ex. 21, Ref. 22.

| Sample No. | Ex. 21 | Ref. 22 |
|---|---|---|
| TiO₂ | | 50 |
| Polystyrene particles | 67 | |
| PEG 9000 | | 25 |
| PVA | 33 | 25 |

### Testing of Enzyme Granules:

1. Whiteness Assessment The whiteness assessment of the enzyme granules was carried out with a spectrophotometric instrument (Konica Minolta CM-2600d) which was calibrated before each measurement using a whiteness standard material. The sample was transferred into a cylindrical sample holder, and the cylinder was closed with a glass lid.

Three individual measurements were carried out at different locations of the enzyme granule sample, and the colorimetric CIE L*a*b values were then calculated. The L-value was a measure for the whiteness of the enzyme granules: the higher the L-value the whiter the enzyme granule.

### 2. Abrasion Stability

Abrasion stability of the enzyme granules was determined with a Heubach Dustmeter Type Ill. In this configuration, the enzyme granules were subjected to the action of four moving steel balls in a cylindrical container. This mechanical action generates dust, which was separated from the enzyme granules by a constant air flow through the sample container, was then collected in a microfilter.

Enzyme granule samples were sieved to 500-1250 μm prior to testing, and the bulk density was determined according to DIN/EN ISO 60. 25 ml of enzyme granules were used in the Heubach test. Measurement settings were 45 rpm rotor speed, air flow 20 L/min and 20 minutes test duration. The total amount of dust was obtained by weighting the filter before and after the test. The enzyme level contained in the dust was determined by a standard protease enzyme assay. The result of the Heubach test was the total mass of enzyme in the dust, normalized to the weight of the enzyme granule sample. Lower values mean a lower risk in enzyme dust generation during handling of the enzyme granules.

### Results:

Table 5 depicts the test results from samples Ex. 1, Ref. 2-4.

| Sample No. | Ex. 1 | Ref. 2 | Ref. 3 | Ref. 4 |
|---|---|---|---|---|
| TiO₂ | | 50 | 50 | 50 |
| Polystyrene particles | 50 | | | |
| PEG 9000 | 50 | 50 | | 25 |
| PEG 12000 | | | 50 | |
| PVA (Mowiol 3-85, from Ku- | | | | 25 |
| raray) | | | | |
| L-value, CIE L*a*b | 82.5 | 81.4 | 80.9 | 80.4 |
| Heubach: μg protease dust/g | 0.37 | 0.74 | 0.85 | 0.81 |

Polystyrene as organic white pigment in the coating has an advantage over TiO₂ in whiteness and abrasion stability of the enzyme granules.

Table 6 depicts the test results from samples Ex. 5-8, Ref. 9-11.

| | Sample No. | | | | | | |
|---|---|---|---|---|---|---|---|
| | Ex. | Ex. | Ex. | Ex. | Ref. | Ref. | Ref. |
| | 5 | 6 | 7 | 8 | 9 | 10 | 11 |
| TiO₂ | | 25 | | | 50 | | |
| Zeolithe | | | | 25 | | 50 | |
| Talkum | | | | | | | 75 |
| Polystyrene particles | 67 | | | | | | |
| Pergopak M | | 25 | 75 | 25 | | | |
| PEG 9000 | 33 | 50 | 25 | 50 | 50 | 50 | 25 |
| L-value, CIE L*a*b | 79.1 | 71.4 | 76.4 | 72.4 | 76.1 | 71.5 | 68.8 |

Except of TiO₂, the use of inorganic white pigments in the coating yields low L-values even at high pigment level in the coating. With Pergopak Mas organic white pigment, the TiO₂ benchmark is matched.

Table 7 depicts the test results from samples Ex. 12-15, Ref. 16-20.

| | Sample No. | | | | | | | | |
|---|---|---|---|---|---|---|---|---|---|
| | Ex. | Ex. | Ex. | Ex. | Ref. | Ref. | Ref. | Ref. | Ref. |
| | 12 | 13 | 14 | 15 | 16 | 17 | 18 | 19 | 20 |
| TiO₂ | | | | | 50 | 67 | 67 | 50 | 67 |
| Zeolithe | | | 25 | | | | | | |
| Polystyrene | 67 | 67 | | 50 | | | | | |
| particles | | | | | | | | | |
| Pergopak M | | | 25 | | | | | | |
| PEG 9000 | | 16.5 | 50 | 25 | 50 | | | 25 | 33 |
| PVA | 33 | 16.5 | | 25 | | 33 | | 25 | |
| Luvitec VA64 | | | | | | | 33 | | |
| (BASF) | | | | | | | | | |
| L-value, CIE | 78.7 | 79.5 | 73.1 | 76.2 | 77.0 | 77.4 | 76.7 | 75.6 | 75.8 |
| L*a*b | | | | | | | | | |
| Heubach: μg | 0.18 | 0.07 | 0.46 | 0.124 | 0.43 | >30 | >30 | 0.66 | |
| protease dust/g | | | | | | | | | |

Whiteness and abrasion resistance of enzyme granules containing polystyrene particles as the pigment in the coating outperforms TiO₂ at the same pigment level in the coating.

Table 8 depicts the test results from samples Ex. 21 and Ref. 22.

| Sample No. | Ex. 21 | Ref. 22 |
|---|---|---|
| TiO₂ | | 50 |
| Polystyrene particles | 67 | |
| PEG 9000 | | 25 |
| PVA | 33 | 25 |
| Heubach: μg protease | 0.035 | 0.057 |
| dust/g | | |

The coating comprising the organic white pigment is less sensitive to abrasion than the coating comprising TiO₂.

1. An enzyme particle comprising a core and a coating, wherein the core comprises at least one enzyme and the coating comprises at least one organic white pigment.

2. The enzyme particle according to claim 1, wherein the enzyme particle is in the form of an enzyme granule.

3. The enzyme particle according to claim 1, wherein the at least one organic white pigment is comprised in the coating in the range of from 10 to 90 wt.-%, based on the total weight of the coating.

4. The enzyme particle according to claim 1, wherein the at least one organic white pigment is in the form of hollow organic particles.

5. The enzyme particle according to claim 1, wherein the at least one white pigment is based on polymers, comprising nonionic ethylenically unsaturated monomers.

6. The enzyme particle according to claim 5, wherein the nonionic ethylenically unsaturated monomer is selected from the group consisting of styrene, acrylonitrile, methacrylamide, methyl methacrylate, ethyl acrylate, ethyl methacrylate, butyl acrylate, butyl methacrylate, 2-ethylhexyl acrylate, 2-ethylhexyl methacrylate, or mixtures thereof.

7. The enzyme particle according to claim 1, wherein the at least one organic white pigment is based on emulsion polymer particles, obtainable by a process for preparing emulsion polymer particles comprising:
a) providing an aqueous emulsion of
i) multi-stage emulsion polymer, comprising a core stage polymer and a sheath stage polymer,
wherein the core stage polymer comprises, as polymerized units, from 5 to 100 percent by weight, based on the weight of the core stage polymer, of hydrophilic ethylenically unsaturated monomer, and from 0 to 95 percent by weight, based on the weight of the core stage polymer, of at least one nonionic ethylenically unsaturated monomer; and wherein the sheath stage polymer comprises, as polymerized units, at least 50 percent by weight of nonionic ethylenically unsaturated monomer;
ii) monomer at a level of at least 0.5 percent by weight based on the weight of the multi-stage emulsion polymer; and
iii) swelling agent;
and b) reducing the level of monomer by at least fifty percent.

8. The enzyme particle according to claim 1, wherein the at least one organic white pigment consists of at least one hollow organic particle, based on emulsion polymer particles, obtainable by sequential polymerization, comprising polymerizing in a sequential polymerization
i) a seed, and
ii) then reacting with a swell-seed comprising 55 to 99.9 wt.-% of one or more nonionic ethylenically unsaturated monomer and 0.1 to 45 wt.-% of one or more ethylenically unsaturated hydrophilic monomer, all based on an overall weight of a core stage polymer comprising both seed and swell-seed,
iii) then polymerizing a first shell comprising 85 to 99.9 wt.-% of one or more than nonionic ethylenically unsaturated monomer and 0.1 to 15 wt.-% of one or more hydrophilic ethylenically unsaturated monomer,
iv) then polymerizing a second shell comprising 85 to 99.9 wt.-% of one or more nonionic ethylenically unsaturated monomer and 0.1 to 15 wt.-% of one or more hydrophilic ethylenically unsaturated monomer,
v) then adding at least one plasticizer monomer having a ceiling temperature below 181° C.,
vi) neutralizing, to a pH of not less than 7.5 or greater, the resultant particles with one or more bases,
vii) then polymerizing a third shell comprising 90 to 99.9 wt.-% of one or more nonionic ethylenically unsaturated monomer and 0.1 to 10 wt.-% of one or more hydrophilic ethylenically unsaturated monomer,
viii) also optionally polymerizing one or more further shells comprising one or more nonionic ethylenically unsaturated monomer and one or more hydrophilic ethylenically unsaturated monomer, wherein
a weight ratio of the swell-seed (ii) to the seed polymer (i) is in a range from 10:1 to 150:1, a weight ratio of the core stage polymer to the first shell (iii) is in a range from 2:1 to 1:5, and a weight ratio of the third shell (vii) to the second shell (iv) is in a range from 1:2 to 1.10.

9. The enzyme particle according to claim 1, wherein the coating is from 5 to 20 wt.-%, of the total weight of the enzyme particle.

10. A washing or cleaning composition, comprising enzyme particles according to claim 1.

11. The washing or cleaning composition of claim 10, wherein the washing or cleaning composition comprises bleach.

12. A food or feed composition, comprising enzyme particles according to claim 1.

13. (canceled)

14. (canceled)

15. (canceled)
